(19) United States
(12) Patent Application Publication
(10) Pub. No.: US 2023/0340083 A1
(43) Pub. Date: Oct. 26, 2023

(54) ANTIBODIES AND METHODS FOR TREATMENT OF INFLUENZA A INFECTION

(71) Applicant: Vir Biotechnology, Inc., San Francisco, CA (US)

(72) Inventors: Davide Corti, Bellinzona (CH); Michael Alexander Schmid, Bellinzona (CH)

(21) Appl. No.: 18/255,779

(22) PCT Filed: Dec. 7, 2021
(86) PCT No.: PCT/US2021/062160

§ 371 Date: Jun. 2, 2023

Related U.S. Application Data
(60) Provisional application No. 63/122,894, filed on Dec. 8, 2020.

(51) Int. Cl. C07K 16/10; C12N 15/63; A61P 31/16

(52) U.S. Cl. CPC C07K 16/1018; C12N 15/63; A61P 31/16; C07K 2317/565; C07K 2317/524

(57) ABSTRACT

The present disclosure provides antibodies that neutralize infection of influenza A virus. The disclosure also provides nucleic acids that encode and immortalized B cells and cultured plasma cells that produce such antibodies. In addition, the disclosure provides the use of the antibodies of the disclosure in prophylaxis and treatment influenza A infection.

The present application claims priority to U.S. Provisional Application No. 63/122,894 filed Dec. 8, 2020, which is incorporated by reference herein.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 930485_431WO_SEQUENCE_LISTING.txt. The text file is 23.9 KB, was created on Dec. 4, 2021, and is being submitted electronically via EFS-Web.

### BACKGROUND

### Technical Field

The disclosure relates to antibodies that potently reduce influenza A infection and to the use of such antibodies. In particular, the disclosure relates to the prophylaxis and treatment of influenza A infection.

### Description of the Related Art

Influenza is an infectious disease, which spreads around the world in yearly outbreaks resulting per year in about three to five million cases of severe illness and about 290,000 to 650,000 respiratory deaths (WHO, Influenza (Seasonal) Fact sheet, Nov. 6, 2018). The most common symptoms include: a sudden onset of fever, cough (usually dry), headache, muscle and joint pain, severe malaise (feeling unwell), sore throat and a runny nose. The incubation period varies between one to four days, although usually the symptoms begin about two days after exposure to the virus. Complications of influenza may include pneumonia, sinus infections, and worsening of previous health problems such as asthma or heart failure, sepsis or exacerbation of chronic underling diseases.

Influenza is caused by influenza virus, an antigenically and genetically diverse group of viruses of the family Orthomyxoviridae that contains a negative-sense, single-stranded, segmented RNA genome. Of the four types of influenza virus (A, B, C and D), three types (A, B and C) affect humans. Influenza type A viruses are the most virulent human pathogens and cause the severest disease. Influenza A viruses can be categorized based on the different subtypes of major surface proteins present: Hemagglutinin (HA) and Neuraminidase (NA). There are at least 18 influenza A subtypes defined by their hemagglutinin (“HA”) proteins. The HAs can be classified into two groups. Group 1 contains H1, H2, H5, H6, H8, H9, H11, H12, H13, H16 and H17 subtypes, and group 2 includes H3, H4, H7, H10, H14 and H15 subtypes. While all subtypes are present in birds, mostly H1, H2 and H3 subtypes cause disease in humans. H5, H7 and H9 subtypes are causing sporadic severe infections in humans and may generate a new pandemic. Influenza A viruses continuously evolve generating new variants, a phenomenon called antigenic drift. As a consequence, antibodies produced in response to past viruses are poorly- or non-protective against new drifted viruses. A consequence is that a new vaccine has to be produced every year against H1 and H3 viruses that are predicted to emerge, a process that is very costly as well as not always efficient. The same applies to the production of a H5 influenza vaccine.

HA is a major surface protein of influenza A virus, which is the main target of neutralizing antibodies that are induced by infection or vaccination. HA is responsible for binding the virus to cells with sialic acid on the membranes, such as cells in the upper respiratory tract or erythrocytes. In addition, HA mediates the fusion of the viral envelope with the endosome membrane, after the pH has been reduced. HA is a homotrimeric integral membrane glycoprotein. The HA trimer is composed of three identical monomers, each made of an intact HA0 single polypeptide chain with HA1 and HA2 regions linked by 2 disulfide bridges. Each HA2 region adopts alpha helical coiled coil structure and primarily forms the “stem” or “stalk” region of HA, while the HA1 region is a small globular domain containing a mix of α/β structures (“head” region of HA). The globular HA head region mediates binding to the sialic acid receptor, while the HA stem mediates the subsequent fusion between the viral and cellular membranes that is triggered in endosomes by the low pH. While the immunodominant HA globular head domain has high plasticity with distinct antigenic sites undergoing constant antigenic drift, the HA stem region is relatively conserved among subtypes. Current influenza vaccines mostly induce an immune response against the immunodominant and variable HA head region, which evolves faster than the stem region of HA (Kirkpatrick E, Qiu X, Wilson P C, Bahl J, Krammer F. The influenza virus hemagglutinin head evolves faster than the stalk domain. Sci Rep. 2018 Jul. 11; 8(1):10432). Therefore, a particular influenza vaccine usually confers protection for no more than a few years and annual re-development of influenza vaccines is required.

To overcome these problems, recently a new class of influenza-neutralizing antibodies that target conserved sites in the HA stem were developed as influenza virus therapeutics. These antibodies targeting the stem region of HA are usually broader neutralizing compared to antibodies targeting the head region of HA. An overview over broadly neutralizing influenza A antibodies is provided in Corti D. and Lanzavecchia A., Broadly neutralizing antiviral antibodies. Annu. Rev. Immunol. 2013; 31:705-742. Okuno et al. immunized mice with influenza virus A/Okuda/57 (H2N2) and isolated a monoclonal antibody (C179) that binds to a conserved conformational epitope in HA2 and neutralizes the Group 1 H2, H1 and H5 subtype influenza A viruses in vitro and in vivo in animal models (Okuno et al., 1993; Smirnov et al., 1999; Smirnov et al., 2000). Further examples of HA-stem region targeting antibodies include CR6261 (Throsby M, van den Brink E, Jongeneelen M, Poon L L M, Alard P, Cornelissen L, et al. (2008) Heterosubtypic Neutralizing Monoclonal Antibodies Cross-Protective against H5N1 and H1N1 Recovered from Human IgM+ Memory B Cells. PLoS ONE 3(12): e3942. https://doi.org/10.1371/journal.pone.0003942; Friesen R H E, Koudstaal W, Koldijk M H, Weverling G J, Brakenhoff J P J, Lenting P J, et al. (2010) New Class of Monoclonal Antibodies against Severe Influenza: Prophylactic and Therapeutic Efficacy in Ferrets. PLoS ONE 5(2): e9106. https://doi.org/10.1371/journal.pone.0009106), F10 (Sui J, Hwang W C, Perez S, Wei G, Aird D, Chen L M, Santelli E, Stec B, Cadwell G, Ali M, Wan H, Murakami A, Yammanuru A, Han T, Cox N J, Bankston L A, Donis R O, Liddington R C, Marasco W A (March 2009). “Structural and functional bases for broad-spectrum neutralization of avian and human influenza A viruses.” Nature Structural & Molecular Biology. 16 (3): 265-73. doi:10.1038/nsmb.1566), CR8020 (Ekiert D C, Friesen R H E, Bhabha G, Kwaks T, Jongeneelen M, et al. 2011. A highly conserved neutralizing epitope on group 2 influenza A viruses. Science 333(6044):843-50), FI6 (Corti D, Voss J, Gamblin S J, Codoni G, Macagno A, et al. 2011. A neutralizing antibody selected from plasma cells that binds to group 1 and group 2 influenza A hemagglutinins. Science 333(6044):850-56), and CR9114 (Dreyfus C, Laursen N S, Kwaks T, Zuijdgeest D, Khayat R, et al. 2012. Highly conserved protective epitopes on influenza B viruses. Science 337(6100):1343-48).

However, antibodies capable of reacting with the HA stem region of both group 1 and 2 subtypes are extremely rare and usually do not show complete coverage of all subtypes. Recently, antibody MEDI8852 was described, which potently neutralizes group 1 and 2 influenza A viruses with unprecedented breadth, being able to neutralize a diverse panel of representative viruses spanning >80 years of antigenic evolution (Kallewaard N L, Corti D, Collins P J, et al. Structure and Function Analysis of an Antibody Recognizing All Influenza A Subtypes. Cell. 2016; 166(3):596-608; Paules, C. I. et al. The Hemagglutinin A Stem Antibody MEDI8852 Prevents and Controls Disease and Limits Transmission of Pandemic Influenza Viruses. J Infect Dis 216, 356-365, https://doi.org/10.1093/infdis/jix292 (2017)). MEDI8852 was shown to bind to a highly conserved epitope that is markedly different from other structurally characterized stem-reactive neutralizing antibodies (Kallewaard N1, Corti D, Collins P J, et al. Structure and Function Analysis of an Antibody Recognizing All Influenza A Subtypes. Cell. 2016; 166(3):596-608).

### BRIEF SUMMARY

In view of the above, it is the object of the present disclosure to provide a novel antibody, which broadly neutralizes influenza A virus and which provides enhanced efficacy in comparison to prior art antibodies.

This object is achieved by means of the subject-matter set out below and in the appended claims.

### BRIEF DESCRIPTION OF THE SEVERAL VIEWS OF THE DRAWINGS

In the following a brief description of the appended figures will be given. The figures are intended to illustrate the present disclosure in more detail. However, they are not intended to limit the subject matter of the disclosure in any way.

FIG. 1 shows, for Example 1, binding of FluAB_wt, FluAB_MLNS, FluAB_GAALIE and FluAB_GAALIE+MLNS to hemagglutinin (HA) as assessed by ELISA against H1N1 HA CA09 (A/California/07/09).

FIGS. 2A and 2B show, for Example 2, (A) neutralization of H1N1 CA09 strain (A/California/07/09) by FluAB_wt, FluAB_MLNS, FluAB_GAALIE and FluAB_GAALIE+MLNS, and (B) neutralization of H3N2 strain A/Aichi/2/68 by FluAB_wt, FluAB_MLNS, and FluAB_GAALIE.

FIG. 3 shows, for Example 3, binding of FluAB_wt, FluAB_MLNS, FluAB_GAALIE and FluAB_GAALIE+MLNS to human FcγRs as assessed by BLI. Binding of FluAB variants to multiple human FcgRs as measured by Octet. His-tagged human FcgRs (FcgRIIa allele H131, FcgRIIa allele R131, FcgRIIa allele F158, FcgRIIIa allele V158 and FcgRIIb) at 2 μg/ml were captured onto anti-penta-His sensors for 6 minutes. FcgRs-loaded sensors were then exposed for 4 minutes to a solution of kinetics buffer (pH 7.1) containing 2 μg/ml of each mAb (left part of the plot) in the presence 1 μg/ml of affiniPure F(ab′)₂ Fragment Goat Anti-Human IgG, F(ab′)₂ fragment specific (to cross-link antibodies through the Fab fragment), followed by a dissociation step in the same buffer for additional 4 minutes (right part of the plot). Association and dissociation profiles were measured in real time as change in the interference pattern using an Octet RED96 (FortéBio).

FIG. 4 shows, for Example 4, binding of FluAB_wt and FluAB_GAALIE to C1q. Binding of FluAB_wt and FluAB_GAALIE to human C1q as measured by Octet. Anti-human Fab (CH1) sensors were used to capture through the Fab fragment the full IgG1 of FluAB_wt and FluAB_GAALIE at 10 μg/ml for 10 minutes. IgG-loaded sensors were then exposed for 4 minutes to a solution of kinetics buffer (pH 7.1) containing 3 μg/ml of purified human C1q (left part of the plot), followed by a dissociation step in the same buffer for additional 4 minutes (right part of the plot).

FIGS. 5A and 5B show, for Example 5, in vitro activation of human FcgRIIIa using receptor-linked activation of a NFAT-mediated luciferase reporter in engineered Jurkat cells. ADCC was tested using well-validated, commercially available ADCC assay in which A549-H1 cells stably transfected to express HA from A/California/07/2009 (H1N1) on the cell surface are used as target cells. Serial dilutions of FluAB wt or Fc variants were added to the HA-expressing cells. A549-H1 cells were incubated together with antibodies at 37° C. for 30 min. Jurkat effector cells (Promega) expressing either FcgRIIIa high affinity allele V158 (5A) or FcgRIIIa low affinity allele F158 (5B) were resuspended in assay buffer and then added to assay plates (effector cells to target cells ratio of 6:1). After incubation at 37° C. for 20 hours, Bio-Glo-TM Luciferase Assay Reagent (Promega) was added, and luminescence was quantified using luminometer (Bio-Tek).

FIG. 6 shows, for Example 5, in vitro activation of human FcgRIIa and human FcgRIIb using receptor-linked activation of a NFAT-mediated luciferase reporter in engineered Jurkat cells. ADCP was tested using well-validated, commercially available ADCP assay in which A549-H1 cells stably transfected to express HA from A/California/07/2009 (H1N1) on the cell surface are used as target cells. Serial dilutions of FluAB Fc wt or variants were added to the HA-expressing cells. A549-H1 cells were incubated together with antibodies at 37° C. for 30 min. Jurkat effector cells (Promega) expressing FcgRIIa high affinity allele H131 or FcgRIIb were resuspended in assay buffer and then added to assay plates (effector cells to target cells ratio of 5:1). After incubation at 37° C. for 21 hours, Bio-Glo-TM Luciferase Assay Reagent (Promega) was added, and luminescence was quantified using luminometer (Bio-Tek).

FIG. 7 shows, for Example 6, in vitro killing of A549 cells expressing H1 HA by human NK cells in the presence of FluAB Fc variants. ADCC was tested using freshly isolated NK cells from one donor previously genotyped for expressing homozygous low (F158) affinity FcγRIIIa. Serial dilutions of FluAB wt or Fc variants mAbs were added to the HA-expressing cells. A549-H1 cells were incubated together with antibodies at 37° C. for 30 min. NK cells were added to assay plates (effector cells to target cells ratio of 6:1) and incubated at 37° C. for 4 hours. Cell death was determined by measuring lactate dehydrogenase (LDH) release.

FIG. 8 shows, for Example 7, binding of FluAB_wt, FluAB_MLNS, FluAB_GAALIE+MLNS and FluAB_GAALIE to human FcRn as assessed by BLI. Binding of human FcRn in solution to immobilized FluAB wt or Fc variants was measured by Octet in real time at pH=6.0. The time point 0 seconds represents switch from base line buffer to buffer containing human FcRn. Time point 300 seconds (red dotted vertical line) represents switch to blank buffer at the corresponding pH. Curves indicate association and dissociation profiles of change in the interference patterns.

FIG. 9 shows, for Example 8, mean and standard deviation (SD) of plasma concentration of FluAB_wt, FluAB_MLNS and FluAB_GAALIE+MLNS following intravenous infusion into cynomolgus monkeys excluding the animals with a confirmed ADA response.

FIG. 10 shows, for Example 8, integrity assessment via comparing total antibody quantification to HA binding. Plasma concentrations of FluAB_MLNS (animals C90142, C90190) or FluAB_GAALIE+MLNS (C90153, C90156) were measured using an anti-CH2 antibody ELISA to quantify total antibody or HA antigen-binding ELISA to determine functionality of the mAbs. Graphs show linear regression between total antibody quantification and HA binding for individual animals at selected time points (days 1, 21, 56, 86, and 113).

FIG. 11 shows, for Example 9, complement-dependent cytotoxicity (CDC) for FluAB_wt, FluAB_MLNS and FluAB_GAALIE+MLNS. Cell death of target cells was quantified by measuring LDH release. The percent specific lysis was determined by applying the following formula: (specific release−spontaneous release)/(maximum release−spontaneous release)×100.

### DETAILED DESCRIPTION

Although the present disclosure is described in detail below, it is to be understood that this disclosure is not limited to the particular methodologies, protocols and reagents described herein as these may vary. It is also to be understood that the terminology used herein is not intended to limit the scope of the present disclosure which will be limited only by the appended claims. Unless defined otherwise, all technical and scientific terms used herein have the same meanings as commonly understood by one of ordinary skill in the art.

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated.

Throughout this specification and the claims which follow, unless the context requires otherwise, the term “comprise,” and variations such as “comprises” and “comprising,” will be understood to imply the inclusion of a stated member, integer or step but not the exclusion of any other non-stated member, integer or step. The term “consist of” is a particular embodiment of the term “comprise,” wherein any other non-stated member, integer or step is excluded. In the context of the present disclosure, the term “comprise” encompasses the term “consist of”. The term “comprising” thus encompasses “including” as well as “consisting” e.g., a composition “comprising” X may consist exclusively of X or may include something additional, e.g., X+Y. The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain) or a protein “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the disclosure.

The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

The word “substantially” does not exclude “completely” e.g., a composition which is “substantially free” from Y may be completely free from Y. Where necessary, the word “substantially” may be omitted from the definition of the disclosure.

The term “about” in relation to a numerical value x means x±10%, for example, x±5%, or x±7%, or x±10%, or x±12%, or x±15%, or x±20%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis, attenuation, amelioration and therapy. In general, an appropriate dose or treatment regimen comprising an antibody or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof. The terms “subject” or “patient” are used interchangeably herein to mean all mammals including humans. Examples of subjects include humans, cows, dogs, cats, horses, goats, sheep, pigs, and rabbits. In some embodiments, the patient is a human.

Doses are often expressed in relation to the bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

The term “specifically binding” and similar reference does not encompass non-specific sticking. “Specifically binding” can refer to an association or union of an antibody to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (K_{d}) of a particular binding interaction with units of M (e.g., 10⁻⁵ M to 10⁻¹³ M).

As used herein, the term “antibody” encompasses various forms of antibodies including, without being limited to, whole antibodies, antibody fragments, human antibodies, chimeric antibodies, humanized antibodies, recombinant antibodies and genetically engineered antibodies (variant or mutant antibodies) as long as the characteristic properties according to the disclosure are retained. In some embodiments, the antibody is a human antibody. In some embodiments, the antibody is a monoclonal antibody. For example, the antibody is a human monoclonal antibody.

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol. 7 (1993) 3340). Human antibodies can also be produced in phage display libraries (Hoogenboom, H. R., and Winter, G., J. Mol. Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). In some embodiments, human monoclonal antibodies are prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5. As used herein, the term “variable region” (variable region of a light chain (VL), variable region of a heavy chain (VH)) denotes each of the pair of light and heavy chains which is involved directly in binding the antibody to the antigen.

Antibodies of the disclosure can be of any isotype (e.g., IgA, IgG, IgM, i.e., an α, γ or μ heavy chain). For example, the antibody is of the IgG type. Within the IgG isotype, antibodies may be IgG1, IgG2, IgG3 or IgG4 subclass, for example IgG1. Antibodies of the disclosure may have a K or a N light chain. In some embodiments, the antibody is of IgG1 type and has a K light chain.

Antibodies according to the present disclosure may be provided in purified form. Typically, the antibody will be present in a composition that is substantially free of other polypeptides, e.g., where less than 90% (by weight), usually less than 60% and more usually less than 50% of the composition is made up of other polypeptides.

Antibodies according to the present disclosure may be immunogenic in human and/or in non-human (or heterologous) hosts, e.g., in mice. For example, the antibodies may have an idiotope that is immunogenic in non-human hosts, but not in a human host. Antibodies of the disclosure for human use include those that cannot be easily isolated from hosts such as mice, goats, rabbits, rats, non-primate mammals, etc., and cannot generally be obtained by humanization or from xeno-mice.

As used herein, a “neutralizing antibody” is one that can neutralize, i.e., prevent, inhibit, reduce, impede or interfere with, the ability of a pathogen to initiate and/or perpetuate an infection in a host. The terms “neutralizing antibody” and “an antibody that neutralizes” or “antibodies that neutralize” are used interchangeably herein. These antibodies can be used alone, or in combination, as prophylactic or therapeutic agents upon appropriate formulation, in association with active vaccination, as a diagnostic tool, or as a production tool as described herein.

As used herein, the term “mutation” relates to a change in the nucleic acid sequence and/or in the amino acid sequence in comparison to a reference sequence, e.g., a corresponding genomic sequence. A mutation, e.g., in comparison to a genomic sequence, may be, for example, a (naturally occurring) somatic mutation, a spontaneous mutation, an induced mutation, e.g., induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g., in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved, e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide. “Isolated” can, in some embodiments, also describe an antibody, polynucleotide, vector, host cell, or composition that is outside of a human body.

The term “gene” means the segment of DNA or RNA involved in producing a polypeptide chain; in certain contexts, it includes regions preceding and following the coding region (e.g., 5′ untranslated region (UTR) and 3′ UTR) as well as intervening sequences (introns) between individual coding segments (exons).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the disclosure is not entitled to antedate such disclosure by virtue of prior disclosure.

It is to be understood that this disclosure is not limited to the particular methodology, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

### Antibodies

The disclosure is based, amongst other findings, on the identification of antibodies that reduce influenza A infection and exhibit enhanced efficacy. One of the crucial mechanisms of action of a therapeutic antibody is the targeted elimination of viruses through recruitment of the immune system. This is typically achieved by interaction of the antibody's Fc domain with Fcγ receptors (FcγRs; FcgammaRs; FcgRs) and/or the complement component C1q. Antibodies of the present disclosure show increased effector functions, namely, an enhanced ability to mediate cellular cytotoxicity functions, such as antibody-dependent cell-mediated cytotoxicity (ADCC) and antibody-dependent cell-mediated phagocytosis (ADCP).

In a first aspect the present disclosure provides an (isolated) antibody comprising the heavy chain CDR1, CDR2, and CDR3 sequences as set forth in SEQ ID NO: 1, SEQ ID NO: 2, and SEQ ID NO: 3, respectively; the light chain CDR1, CDR2, and CDR3 sequences as set forth in SEQ ID NO: 4, SEQ ID NO: 5, and SEQ ID NO: 6, respectively; and the mutations G236A, A330L and I332E in the constant region of the heavy chain.

In some embodiments, the antibody does not comprise the mutation S239D in the constant region of the heavy chain. In some embodiments, the antibody comprises a S at position 239 in the constant region of the heavy chain.

In general, the antibody according to the present disclosure, typically comprises (at least) three complementarity determining regions (CDRs) on a heavy chain and (at least) three CDRs on a light chain. In general, complementarity determining regions (CDRs) are the hypervariable regions present in heavy chain variable domains and light chain variable domains. Typically, the CDRs of a heavy chain and the connected light chain of an antibody together form the antigen receptor. Usually, the three CDRs (CDR1, CDR2, and CDR3) are arranged non-consecutively in the variable domain. Since antigen receptors are typically composed of two variable domains (on two different polypeptide chains, i.e., heavy and light chain), there are six CDRs for each antigen receptor (heavy chain: CDRH1, CDRH2, and CDRH3; light chain: CDRL1, CDRL2, and CDRL3). A single antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a chain (or each chain, respectively) may be composed of four framework regions, separated by three CDRs.

Numbering of CDR and framework regions may be according to any known method or scheme, such as the Kabat, Chothia, EU, IMGT, Contact, North, Martin, and AHo numbering schemes (see, e.g., Kabat et al., Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Plückthun, J. Mol. Bio. 309:657-670 (2001); North et al. J Mol Biol. (2011) 406:228-56; doi:10.1016/j.jmb.2010.10.030; Abhinandan and Martin, Mol Immunol. (2008) 45:3832-9. 0.1016/j.molimm. 2008.05.022). The antibody and CDR numbering systems of these references are incorporated herein by reference. Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme. In certain embodiments, an antibody is provided that comprises CDRs of a VH sequence according to SEQ ID NO.:7, and CDRs if a VL sequence according to SEQ ID NO.:8, in accordance with any known CDR numbering method, including the Kabat, Chothia, EU, IMGT, Martin (Enhanced Chothia), Contact, and AHo numbering methods. In certain embodiments, CDRs are according to the IMGT numbering method. In certain embodiments, CDRs are according to the antibody numbering method developed by the Chemical Computing Group (CCG); e.g., using Molecular Operating Environment (MOE) software (www.chemcomp.com). In some embodiments, the CDRs are according to the Kabat, Chothia, AhHo, or North numbering system.

The sequences of the heavy chains and light chains of exemplary antibodies of the disclosure, comprising three different CDRs on the heavy chain and three different CDRs on the light chain were determined. The position of the CDR amino acids are defined, unless otherwise indicated, according to the Kabat system as set forth in Kabat et al., (1991) Sequences of Proteins of Immunological Interest, 5th Ed. Public Health Service, National Institutes of Health, Bethesda, MD.

Typically, the antibody of the disclosure binds to hemagglutinin of an influenza A virus. Thereby, the antibody of the disclosure can neutralize infection of influenza A virus. The antibody according to the present disclosure binds, through CDRs, to the same epitope of the influenza A virus hemagglutinin (IAV HA) stem region as MEDI8852 (Kallewaard N L, Corti D, Collins P J, et al. Structure and Function Analysis of an Antibody Recognizing All Influenza A Subtypes. Cell. 2016; 166(3):596-608), thereby providing the same broad protection against various influenza A serotypes of all influenza A subtypes.

To study and quantitate virus infectivity (or “neutralization”) in the laboratory the person skilled in the art knows various standard “neutralization assays.” For a neutralization assay animal viruses are typically propagated in cells and/or cell lines. For example, in a neutralization assay cultured cells may be incubated with a fixed amount of influenza A virus (IAV) in the presence (or absence) of the antibody to be tested. As a readout for example flow cytometry may be used. Alternatively, also other readouts are conceivable.

The antibody of the present disclosure includes three mutations in the constant region of the heavy chain (in the CH2 region): G236A, A330L and I332E. As outlined above, in some embodiments, the antibody does not comprise the mutation S239D in the constant region of the heavy chain. In the context of the constant region of an antibody, the amino acid positions have been numbered herein according to the art-recognized EU numbering system. The EU index or EU index as in Kabat or EU numbering refers to the numbering of the EU antibody (Edelman G M, Cunningham B A, Gall W E, Gottlieb P D, Rutishauser U, Waxdal M J. The covalent structure of an entire gammaG immunoglobulin molecule. Proc Natl Acad Sci USA. 1969; 63(1):78-85; Kabat E. A., National Institutes of Health (U.S.) Office of the Director, “Sequences of Proteins of Immunological Interest,” 5^{th} edition, Bethesda, MD: U.S. Dept. of Health and Human Services, Public Health Service, National Institutes of Health, 1991, hereby entirely incorporated by reference). As shown in the enclosed Examples, the three mutations G236A, A330L and I332E result in increased effector functions of the antibody.

In some embodiments, the antibody also comprises a half-life increasing mutation in the constant region of the heavy chain. In general, the expression “half-life increasing mutation” may refer to a single mutation, such as a single amino acid substitution, or a group of mutations, such as a group of (i.e., more than one, e.g., 2, 3, 4, 5, 6, 7, 8, 9, 10 or more) amino acid substitutions, which mediate increased half-life of the antibody. Examples of such modifications include, but are not limited to, substitutions of at least one amino acid from the heavy chain constant region selected from the group consisting of amino acid residues 250, 314, and 428. Further examples of such half-life extending Fc modifications are described in Wang Y, Tian Z, Thirumalai D, Zhang X. Neonatal Fc receptor (FcRn): a novel target for therapeutic antibodies and antibody engineering. J Drug Target. 2014 May; 22(4):269-78, the half-life increasing Fc modifications of which are incorporated herein by reference. In some embodiments, a half-life increasing mutation comprises any one or more of: M428L; N434S; N434H; N434A; N434S; M252Y; S254T; T256E; T250Q; P257I Q311I; D376V; T307A; E380A (EU numbering). In certain embodiments, a half-life increasing mutation comprises M252Y/S254T/T256E. In certain embodiments, a half-life increasing mutation comprises T250Q/M428L. In certain embodiments, a half-life increasing mutation comprises P257I/Q311I. In certain embodiments, a half-life increasing mutation comprises P257I/N434H. In certain embodiments, a half-life increasing mutation comprises D376V/N434H. In certain embodiments, a half-life increasing comprises T307A/E380A/N434A. In some embodiments, the antibody comprises the mutation(s) M428L and/or N434S in the heavy chain constant region (CH3 region).

In particular, the mutations G236A, A330L and I332E in the constant region of the heavy chain of the antibody of the disclosure do not compromise the half-life increasing effect of respective mutations in the constant region, as shown in the enclosed Examples.

In some embodiments, the antibody of the disclosure is a human antibody. In some embodiments, the antibody of the disclosure is a monoclonal antibody. For example, the antibody of the disclosure is a human monoclonal antibody.

Antibodies of the disclosure can be of any isotype (e.g., IgA, IgG, IgM, i.e., an α, γ or μ heavy chain). For example, the antibody is of the IgG type. Within the IgG isotype, antibodies may be IgG1, IgG2, IgG3 or IgG4 subclass, for example IgG1. Antibodies of the disclosure may have a K or a X light chain. In some embodiments, the antibody has a kappa (κ) light chain. In some embodiments, the antibody is of IgG1 type and has a K light chain.

In some embodiments, the antibody is of the human IgG1 type. The antibody may be of any allotype. The term “allotype” refers to the allelic variation found among the IgG subclasses. For example, the antibody may be of the G1 ml (or G1m(a)) allotype, of the G1m2 (or G1m(x)) allotype, of the G1m3 (or G1m(f)) allotype, and/or of the G1m17 (or Gm(z)) allotype. The G1m3 and G1m17 allotypes are located at the same position in the CH1 domain (position 214 according to EU numbering). G1m3 corresponds to R214 (EU), while G1m17 corresponds to K214 (EU). The G1 ml allotype is located in the CH3 domain (at positions 356 and 358 (EU)) and refers to the replacements E356D and M358L. The G1m2 allotype refers to a replacement of the alanine in position 431 (EU) by a glycine. The G1 ml allotype may be combined, for example, with the G1m3 or the G1m17 allotype. In some embodiments, the antibody is of the allotype G1m3 with no G1 ml (G1m3,-1). In some embodiments, the antibody is of the G1m17,1 allotype. In some embodiments, the antibody is of the G1m3,1 allotype. In some embodiments, the antibody is of the allotype G1m17 with no G1 ml (G1m17,-1). Optionally, these allotypes may be combined (or not combined) with the G1m2, G1m27 or G1m28 allotype. For example, the antibody may be of the G1m17,1,2 allotype.

In some embodiments, the antibody of the disclosure comprises a heavy chain variable region comprising an amino acid sequence having 70% or more (i.e., 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having at least 70% identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 6, respectively) are maintained.

Sequence identity is usually calculated with regard to the full length of the reference sequence (i.e., the sequence recited in the application). Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=11 and gap extension penalty=1].

A “sequence variant” has an altered sequence in which one or more of the amino acids in the reference sequence is/are deleted or substituted, and/or one or more amino acids is/are inserted into the sequence of the reference amino acid sequence. As a result of the alterations, the amino acid sequence variant has an amino acid sequence which is at least 70% identical to the reference sequence. Variant sequences which are at least 70% identical have no more than 30 alterations, i.e., any combination of deletions, insertions or substitutions, per 100 amino acids of the reference sequence.

In general, while it is possible to have non-conservative amino acid substitutions, the substitutions are usually conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g., alanine, valine, leucine and isoleucine, with another; substitution of one hydoxyl-containing amino acid, e.g., serine and threonine, with another; substitution of one acidic residue, e.g., glutamic acid or aspartic acid, with another; replacement of one amide-containing residue, e.g., asparagine and glutamine, with another; replacement of one aromatic residue, e.g., phenylalanine and tyrosine, with another; replacement of one basic residue, e.g., lysine, arginine and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

In some embodiments, the antibody of the disclosure comprises a heavy chain variable region comprising an amino acid sequence having 75% or more (i.e., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having at least 75% identity to SEQ ID NO: 8, wherein the CDR sequences as defined above are maintained. In some embodiments, the antibody of the disclosure comprises a heavy chain variable region comprising an amino acid sequence having 80% or more (i.e., 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having at least 80% identity to SEQ ID NO: 8, wherein the CDR sequences as defined above are maintained. In some embodiments, the antibody of the disclosure comprises a heavy chain variable region comprising an amino acid sequence having 85% or more (i.e., 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having at least 85% identity to SEQ ID NO: 8, wherein the CDR sequences as defined above are maintained. In some embodiments, the antibody of the disclosure comprises a heavy chain variable region comprising an amino acid sequence having 90% or more (i.e., 91%, 92%, 93%, 94%, 95%, 96%, 97%, 98%, 99% or more) identity to SEQ ID NO: 7 and a light chain variable region comprising the amino acid sequence having at least 90% identity to SEQ ID NO: 8, wherein the CDR sequences as defined above are maintained. In some embodiments, the antibody of the disclosure comprises a heavy chain variable region comprising an amino acid sequence having 95% or more (i.e., 96%, 97%, 98%, 99% or more) identity to SEQ ID NO: 7 and a light chain variable region comprising the amino acid sequence having at least 95% identity to SEQ ID NO: 8, wherein the CDR sequences as defined above are maintained.

In some embodiments, the antibody of the disclosure comprises a heavy chain variable region comprising an amino acid sequence as set forth in SEQ ID NO: 7 and a light chain variable region comprising the amino acid sequence as set forth in SEQ ID NO: 8, wherein the CDR sequences as defined above are maintained.

In general, it is possible that the antibody of the disclosure comprises one or more further mutations (in addition to the mutations G236A, A330L and I332E and, optionally, a half-life increasing mutation, such as M428L and N434S) in the Fc region (e.g., in the CH2 or CH3 region). However, in some embodiments, the antibody of the disclosure does not comprise any further mutation in addition to G236A, A330L and I332E in its CH2 region (in comparison to the respective wild-type CH2 region). In some embodiments, the antibody of the disclosure does not comprise (i) any mutation in its CH3 region; or (ii) any further mutation in addition to M428L and N434S in its CH3 region (in comparison to the respective wild-type CH3 region). In some embodiments, the antibody of the disclosure does not comprise any further mutation in addition to G236A, A330L and I332E and, optionally, M428L and N434S, in its Fc region (in comparison to the respective wild-type Fc region). As used herein, the term “wild-type” refers to the reference sequence, for example as occurring in nature. As a specific example, the term “wild-type” may refer to the sequence with the highest prevalence occurring in nature.

In some embodiments, the antibody of the disclosure comprises a heavy chain comprising an amino acid sequence as set forth in SEQ ID NO: 9 and a light chain comprising an amino acid sequence as set forth in SEQ ID NO: 10. For example, the antibody of the disclosure may have a heavy chain consisting of an amino acid sequence as set forth in SEQ ID NO: 9 and a light chain consisting of an amino acid sequence as set forth in SEQ ID NO: 10.

In some embodiments, the antibody of the disclosure comprises a heavy chain comprising an amino acid sequence as set forth in SEQ ID NO: 13 and a light chain comprising an amino acid sequence as set forth in SEQ ID NO: 10. For example, the antibody of the disclosure may have a heavy chain consisting of an amino acid sequence as set forth in SEQ ID NO: 13 and a light chain consisting of an amino acid sequence as set forth in SEQ ID NO: 10.

In some embodiments, an antibody is provided that comprises a heavy chain comprising or consisting of the amino acid sequence of SEQ ID NO:9 or 13 and a light chain comprising or consisting of SEQ ID NO:10.

In some embodiments, an antibody is provided that comprises two heavy chains, each comprising or consisting of the amino acid sequence of SEQ ID NO:9 or 13, and two light chains, each comprising or consisting of SEQ ID NO:10.

Antibodies of the disclosure also include hybrid antibody molecules that comprise the six CDRs from an antibody of the disclosure as defined above and one or more CDRs from another antibody to the same or a different epitope or antigen. In some embodiments, such hybrid antibodies comprise six CDRs from an antibody of the disclosure and six CDRs from another antibody to a different epitope or antigen.

Variant antibodies are also included within the scope of the disclosure. Thus, variants of the sequences recited in the application are also included within the scope of the disclosure. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Antibodies of the disclosure may be provided in purified form. Typically, the antibody will be present in a composition that is substantially free of other polypeptides, e.g., where less than 90% (by weight), usually less than 60% and more usually less than 50% of the composition is made up of other polypeptides.

Antibodies of the disclosure may be immunogenic in non-human (or heterologous) hosts, e.g., in mice. In particular, the antibodies may have an idiotope that is immunogenic in non-human hosts, but not in a human host. In particular, antibodies of the disclosure for human use include those that cannot be easily isolated from hosts such as mice, goats, rabbits, rats, non-primate mammals, etc., and cannot generally be obtained by humanization or from xeno-mice.

### Nucleic Acids

In another aspect, the disclosure also provides a nucleic acid molecule comprising a polynucleotide encoding the antibody according to the present disclosure as described above, or a portion thereof (e.g., a CDR, a VH, a VL, a heavy chain, or a light chain). Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA. In certain embodiments, the nucleic acid molecule comprises DNA or RNA, wherein the RNA optionally comprises messenger RNA (mRNA). The nucleic acid molecule may encode the light chain and/or the heavy chain of the antibody of the disclosure. In other words, the light chain and the heavy chain of the antibody may be encoded by the same nucleic acid molecule (e.g., in bicistronic manner). Alternatively, the light chain and the heavy chain of the antibody may be encoded by distinct nucleic acid molecules.

In some embodiments, the nucleic acid molecule comprises a modified nucleoside, a cap-1 structure, a cap-2 structure, or any combination thereof. In certain embodiments, the polynucleotide comprises a pseudouridine, a N6-methyladenonsine, a 5-methylcytidine, a 2-thiouridine, or any combination thereof. In some embodiments, the pseudouridine comprises NI-methylpseudouridine.

Due to the redundancy of the genetic code, the present disclosure also comprises sequence variants of nucleic acid sequences, which encode the same amino acid sequences. The polynucleotide encoding the antibody (or the complete nucleic acid molecule) may be optimized for expression of the antibody. For example, codon optimization of the nucleotide sequence may be used to improve the efficiency of translation in expression systems for the production of the antibody. Moreover, the nucleic acid molecule may comprise heterologous elements (i.e., elements, which in nature do not occur on the same nucleic acid molecule as the coding sequence for the (heavy or light chain of) an antibody. For example, a nucleic acid molecule may comprise a heterologous promotor, a heterologous enhancer, a heterologous UTR (e.g., for optimal translation/expression), a heterologous Poly-A-tail, and the like.

A nucleic acid molecule is a molecule comprising nucleic acid components. The term nucleic acid molecule usually refers to DNA or RNA molecules. It may be used synonymous with the term “polynucleotide,” i.e., the nucleic acid molecule may consist of a polynucleotide encoding the antibody. Alternatively, the nucleic acid molecule may also comprise further elements in addition to the polynucleotide encoding the antibody. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc., DNA or RNA molecules.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g., labels) or can introduce tags (e.g., for purification purposes). Alternatively, a mutation in a nucleic acid sequence may be “silent,” i.e., not reflected in the amino acid sequence due to the redundancy of the genetic code. In general, mutations can be introduced in specific sites or can be introduced at random, followed by selection (e.g., molecular evolution). For instance, one or more nucleic acids encoding any of the light or heavy chains of an (exemplary) antibody of the disclosure can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the polynucleotide encoding the antibody, (or the (complete) nucleic acid molecule) may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, JCat: a novel tool to adapt codon usage of a target gene to its potential expression host. Nucleic Acids Res. 2005 Jul. 1; 33(Web Server issue):W526-31; or, for example, Genscript's OptimumGene™ algorithm (as described in US 2011/0081708 A1).

The present disclosure also provides a combination of a first and a second nucleic acid molecule, wherein the first nucleic acid molecule comprises a polynucleotide encoding the heavy chain of the antibody of the present disclosure; and the second nucleic acid molecule comprises a polynucleotide encoding the corresponding light chain of the same antibody. The above description regarding the (general) features of the nucleic acid molecule of the disclosure applies accordingly to the first and second nucleic acid molecule of the combination. For example, one or both of the polynucleotides encoding the heavy and/or light chain(s) of the antibody may be codon-optimized.

In certain embodiments, polynucleotide encoding an antibody heavy chain comprises a polynucleotide having at least 50% (i.e., 50%, 55%, 60%, 65%, 70%, 75%, 80%, 85%, 90%, 91%, 92%, 93%, 94%, 95%, 96%, 97%, 98%, 99%, or 100%) identity to the polynucleotide sequence according to SEQ ID NO:14. In some embodiments, a polynucleotide encoding an antibody heavy chain comprises or consists of the polynucleotide sequence of SEQ ID NO:14. In certain embodiments, polynucleotide encoding an antibody light chain comprises a polynucleotide having at least 50% (i.e., 50%, 55%, 60%, 65%, 70%, 75%, 80%, 85%, 90%, 91%, 92%, 93%, 94%, 95%, 96%, 97%, 98%, 99%, or 100%) identity to the polynucleotide sequence according to SEQ ID NO:15. In some embodiments, a polynucleotide encoding an antibody light chain comprises or consists of the polynucleotide sequence of SEQ ID NO:15. In some embodiments, a polynucleotide encoding an antibody heavy chain comprises or consists of the polynucleotide sequence of SEQ ID NO:14, and a polynucleotide encoding an antibody light chain comprises or consists of the polynucleotide sequence of SEQ ID NO:15.

In any of the presently disclosed embodiments, the polynucleotide can comprise deoxyribonucleic acid (DNA) or ribonucleic acid (RNA). In some embodiments, the RNA comprises messenger RNA (mRNA).

### Vector

Further included within the scope of the disclosure are vectors, for example, expression vectors, comprising a nucleic acid molecule according to the present disclosure. Usually, a vector comprises a nucleic acid molecule as described above.

The present disclosure also provides a combination of a first and a second vector, wherein the first vector comprises a first nucleic acid molecule as described above (for the combination of nucleic acid molecules) and the second vector comprises a second nucleic acid molecule as described above (for the combination of nucleic acid molecules).

A vector is usually a (recombinant) nucleic acid molecule, which does not occur in nature. Accordingly, the vector may comprise heterologous elements (i.e., sequence elements of different origin in nature). For example, the vector may comprise a multi cloning site, a heterologous promotor, a heterologous enhancer, a heterologous selection marker (to identify cells comprising said vector in comparison to cells not comprising said vector) and the like. A vector in the context of the present disclosure is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a (heavy and/or light chain of a) desired antibody according to the present disclosure. An expression vector may be used for production of expression products such as RNA, e.g., mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present disclosure may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

A vector can comprise any one or more of the vectors disclosed herein. In particular embodiments, a vector is provided that comprises a DNA plasmid construct encoding the antibody or a portion thereof (e.g., so-called “DMAb”; see, e.g., Muthumani et al., J Infect Dis. 214(3):369-378 (2016); Muthumani et al., Hum Vaccin Immunother 9:2253-2262 (2013)); Flingai et al., Sci Rep. 5:12616 (2015); and Elliott et al., NPJ Vaccines 18 (2017), which antibody-coding DNA constructs and related methods of use, including administration of the same, are incorporated herein by reference). In certain embodiments, a DNA plasmid construct comprises a single open reading frame encoding a heavy chain and a light chain (or a VH and a VL) of the antibody, wherein the sequence encoding the heavy chain and the sequence encoding the light chain are optionally separated by polynucleotide encoding a protease cleavage site and/or by a polynucleotide encoding a self-cleaving peptide. In some embodiments, the substituent components of the antibody are encoded by a polynucleotide comprised in a single plasmid. In other embodiments, the substituent components of the antibody are encoded by a polynucleotide comprised in two or more plasmids (e.g., a first plasmid comprises a polynucleotide encoding a heavy chain, VH, or VH+CH1, and a second plasmid comprises a polynucleotide encoding the cognate light chain, VL, or VL+CL). In certain embodiments, a single plasmid comprises a polynucleotide encoding a heavy chain and/or a light chain from two or more antibodies of the present disclosure. An exemplary expression vector is pVax1, available from Invitrogen®. A DNA plasmid of the present disclosure can be delivered to a subject by, for example, electroporation (e.g., intramuscular electroporation), or with an appropriate formulation (e.g., hyaluronidase).

In some embodiments, method is provided that comprises administering to a subject a first polynucleotide (e.g., mRNA) encoding an antibody heavy chain, a VH, or a Fd (VH+CH1), and administering to the subject a second polynucleotide (e.g., mRNA) encoding the cognate antibody light chain, VL, or VL+CL.

In some embodiments, a polynucleotide (e.g., mRNA) is provided that encodes a heavy chain and a light chain of an antibody. In some embodiments, a polynucleotide (e.g., mRNA) is provided that encodes two heavy chains and two light chains of an antibody. See, e.g. Li, J Q., Zhang, Z R., Zhang, H Q. et al. Intranasal delivery of replicating mRNA encoding neutralizing antibody against SARS-CoV-2 infection in mice. Sig Transduct Target Ther 6, 369 (2021). https://doi.org/10.1038/s41392-021-00783-1, the antibody-encoding mRNA constructs, vectors, and related techniques of which are incorporated herein by reference. In some embodiments, a polynucleotide is delivered to a subject via an alphavirus replicon particle (VRP) delivery system. In some embodiments, a replicon comprises a modified VEEV replicon comprising two subgenomic promoters. In some embodiments, a polynucleotide or replicon can translate simultaneously the heavy chain (or VH, or VH+1) and the light chain (or VL, or VL+CL) of an antibody. In some embodiments, a method is provided that comprises delivering to a subject such a polynucleotide or replicon. In a further aspect, the present disclosure also provides a host cell expressing an antibody according to the present disclosure; or comprising or containing a vector or polynucleotide according the present disclosure.

### Cells

In a further aspect, the present disclosure also provides cell expressing the antibody according to the present disclosure; and/or comprising the vector according the present disclosure.

Examples of such cells include but are not limited to, eukaryotic cells, e.g., yeast cells, animal cells or plant cells or prokaryotic cells, including E. coli. In some embodiments, the cells are mammalian cells, such as a mammalian cell line. Examples include human cells, CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

Insect cells useful expressing a binding protein of the present disclosure are known in the art and include, for example, Spodoptera frugipera Sf9 cells, Trichoplusia ni BTI-TN5B1-4 cells, and Spodoptera frugipera SfSWT01 “Mimic™” cells. See, e.g., Palmberger et al., J. Biotechnol. 153(3-4):160-166 (2011). Numerous baculoviral strains have been identified which may be used in conjunction with insect cells, particularly for transfection of Spodoptera frugiperda cells.

Eukaryotic microbes such as filamentous fungi or yeast are also suitable hosts for cloning or expressing protein-encoding vectors, and include fungi and yeast strains with “humanized” glycosylation pathways, resulting in the production of an antibody with a partially or fully human glycosylation pattern. See Gerngross, Nat. Biotech. 22:1409-1414 (2004); Li et al., Nat. Biotech. 24:210-215 (2006).

Plant cells can also be utilized as hosts for expressing an antibody of the present disclosure. For example, PLANTIBODIES™ technology (described in, for example, U.S. Pat. Nos. 5,959,177; 6,040,498; 6,420,548; 7,125,978; and 6,417,429) employs transgenic plants to produce antibodies.

In certain embodiments, the host cell comprises a mammalian cell. In particular embodiments, the host cell is a CHO cell, a HEK293 cell, a PER.C6 cell, a Y0 cell, a Sp2/0 cell, a NS0 cell, a human liver cell, a myeloma cell, or a hybridoma cell.

The cell may be transfected with a vector according to the present disclosure, for example with an expression vector. The term “transfection” refers to the introduction of nucleic acid molecules, such as DNA or RNA (e.g., mRNA) molecules, into cells, e.g., into eukaryotic or prokaryotic cells. In the context of the present disclosure, the term “transfection” encompasses any method known to the skilled person for introducing nucleic acid molecules into cells, such as into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral.

Moreover, the cells of the present disclosure may be transfected stably or transiently with the vector according to the present disclosure, e.g., for expressing the antibody according to the present disclosure. In some embodiments, the cells are stably transfected with the vector according to the present disclosure encoding the antibody according to the present disclosure. In other embodiments, the cells are transiently transfected with the vector according to the present disclosure encoding the antibody according to the present disclosure.

Accordingly, the present disclosure also provides a recombinant host cell, which heterologously expresses the antibody of the disclosure. For example, the cell may be of another species than the antibody (e.g., CHO cells expressing human antibodies). In some embodiments, the cell type of the cell does not express (such) antibodies in nature. Moreover, the host cell may impart a post-translational modification (PTM; e.g., glycosylation) on the antibody that is not present in their native state or abolish a PTM on the antibody that is present in the antibody's native state. Such an additional or removed PTM may result in a functional difference (e.g., reduced immunogenicity). Accordingly, the antibody of the disclosure may have a post-translational modification, which is distinct from the naturally produced antibody (e.g., an antibody of an immune response in a human).

### Production of Antibodies

Antibodies according to the disclosure can be made by any method known in the art. For example, the general methodology for making monoclonal antibodies using hybridoma technology is well known (Kohler, G. and Milstein, C, 1975; Kozbar et al. 1983). In some embodiments, the alternative EBV immortalization method described in WO2004/076677 is used.

In some embodiments, the method as described in WO 2004/076677, which is incorporated herein by reference, is used. In this method B cells producing the antibody of the disclosure are transformed with EBV and a polyclonal B cell activator. Additional stimulants of cellular growth and differentiation may optionally be added during the transformation step to further enhance the efficiency. These stimulants may be cytokines such as IL-2 and IL-15. In one aspect, IL-2 is added during the immortalization step to further improve the efficiency of immortalization, but its use is not essential. The immortalized B cells produced using these methods can then be cultured using methods known in the art and antibodies isolated therefrom.

Another exemplified method is described in WO 2010/046775. In this method plasma cells are cultured in limited numbers, or as single plasma cells in microwell culture plates. Antibodies can be isolated from the plasma cell cultures. Further, from the plasma cell cultures, RNA can be extracted and PCR can be performed using methods known in the art. The VH and VL regions of the antibodies can be amplified by RT-PCR (reverse transcriptase PCR), sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

Standard techniques of molecular biology may be used to prepare DNA sequences encoding the antibodies of the present disclosure. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of nucleic acid sequences encoding the antibody molecules of the present disclosure. Eukaryotic, e.g., mammalian, host cell expression systems may be used for production of antibody molecules, such as complete antibody molecules. Suitable mammalian host cells include, but are not limited to, CHO, HEK293T, PER.C6, NS0, myeloma or hybridoma cells. In other embodiments, prokaryotic cells, including, but not limited to, E. coli, may be used for the expression of nucleic acid sequences encoding the antibody molecules of the present disclosure.

The present disclosure also provides a process for the production of an antibody molecule according to the present disclosure comprising culturing a (heterologous) host cell comprising a vector encoding a nucleic acid of the present disclosure under conditions suitable for expression of protein from DNA encoding the antibody molecule of the present disclosure, and isolating the antibody molecule.

For production of the antibody comprising both heavy and light chains, a cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Antibodies according to the disclosure may be produced by (i) expressing a nucleic acid sequence according to the disclosure in a host cell, e.g., by use of a vector according to the present disclosure, and (ii) isolating the expressed antibody product. Additionally, the method may include (iii) purifying the isolated antibody. Transformed B cells and cultured plasma cells may be screened for those producing antibodies of the desired specificity or function.

The screening step may be carried out by any immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function, e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

The immortalized B cell clones or the transfected host-cells of the disclosure can be used in various ways, e.g., as a source of monoclonal antibodies, as a source of nucleic acid (DNA or mRNA) encoding a monoclonal antibody of interest, for research, etc.

The disclosure also provides a composition comprising immortalized B memory cells or transfected host cells that produce antibodies according to the present disclosure.

The immortalized B cell clone or the cultured plasma cells of the disclosure may also be used as a source of nucleic acid for the cloning of antibody genes for subsequent recombinant expression. Expression from recombinant sources may be more common for pharmaceutical purposes than expression from B cells or hybridomas, e.g., for reasons of stability, reproducibility, culture ease, etc.

Thus the disclosure also provides a method for preparing a recombinant cell, comprising the steps of: (i) obtaining one or more nucleic acids (e.g., heavy and/or light chain mRNAs) from the B cell clone or the cultured plasma cells that encodes the antibody of interest; (ii) inserting the nucleic acid into an expression vector and (iii) transfecting the vector into a (heterologous) host cell in order to permit expression of the antibody of interest in that host cell.

Similarly, the disclosure also provides a method for preparing a recombinant cell, comprising the steps of: (i) sequencing nucleic acid(s) from the B cell clone or the cultured plasma cells that encodes the antibody of interest; and (ii) using the sequence information from step (i) to prepare nucleic acid(s) for insertion into a host cell in order to permit expression of the antibody of interest in that host cell. The nucleic acid may, but need not, be manipulated between steps (i) and (ii) to introduce restriction sites, to change codon usage, and/or to optimize transcription and/or translation regulatory sequences.

Furthermore, the disclosure also provides a method of preparing a transfected host cell, comprising the step of transfecting a host cell with one or more nucleic acids that encode an antibody of interest, wherein the nucleic acids are nucleic acids that were derived from an immortalized B cell clone or a cultured plasma cell of the disclosure. Thus the procedures for first preparing the nucleic acid(s) and then using it to transfect a host cell can be performed at different times by different people in different places (e.g., in different countries).

These recombinant cells of the disclosure can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

Methods for obtaining and sequencing immunoglobulin genes from B cells or plasma cells are well known in the art (e.g., see Chapter 4 of Kuby Immunology, 4th edition, 2000).

The transfected host cell may be a eukaryotic cell, including yeast and animal cells, particularly mammalian cells (e.g., CHO cells, NS0 cells, human cells such as PER.C6 or HKB-11 cells, myeloma cells, or a human liver cell), as well as plant cells. In some embodiments, the transfected host cell may a prokaryotic cell, including E. coli. In some embodiments, the transfected host cell is a mammalian cell, such as a human cell. In some embodiments, expression hosts can glycosylate the antibody of the disclosure, particularly with carbohydrate structures that are not themselves immunogenic in humans. In some embodiments the transfected host cell may be able to grow in serum-free media. In further embodiments the transfected host cell may be able to grow in culture without the presence of animal-derived products. The transfected host cell may also be cultured to give a cell line.

The disclosure also provides a method for preparing one or more nucleic acid molecules (e.g., heavy and light chain genes) that encode an antibody of interest, comprising the steps of: (i) preparing an immortalized B cell clone or culturing plasma cells according to the disclosure; (ii) obtaining from the B cell clone or the cultured plasma cells nucleic acid that encodes the antibody of interest. Further, the disclosure provides a method for obtaining a nucleic acid sequence that encodes an antibody of interest, comprising the steps of: (i) preparing an immortalized B cell clone or culturing plasma cells according to the disclosure; (ii) sequencing nucleic acid from the B cell clone or the cultured plasma cells that encodes the antibody of interest.

The disclosure further provides a method of preparing nucleic acid molecule(s) that encode an antibody of interest, comprising the step of obtaining the nucleic acid that was obtained from a transformed B cell clone or cultured plasma cells of the disclosure. Thus the procedures for first obtaining the B cell clone or the cultured plasma cell, and then obtaining nucleic acid(s) from the B cell clone or the cultured plasma cells can be performed at different times by different people in different places (e.g., in different countries).

The disclosure also comprises a method for preparing an antibody (e.g., for pharmaceutical use) according to the present disclosure, comprising the steps of: (i) obtaining and/or sequencing one or more nucleic acids (e.g., heavy and light chain genes) from the selected B cell clone or the cultured plasma cells expressing the antibody of interest; (ii) inserting the nucleic acid(s) into or using the nucleic acid(s) sequence(s) to prepare an expression vector; (iii) transfecting a host cell that can express the antibody of interest; (iv) culturing or sub-culturing the transfected host cells under conditions where the antibody of interest is expressed; and, optionally, (v) purifying the antibody of interest.

The disclosure also provides a method of preparing the antibody of interest comprising the steps of: culturing or sub-culturing a transfected host cell population, e.g., a stably transfected host cell population, under conditions where the antibody of interest is expressed and, optionally, purifying the antibody of interest, wherein said transfected host cell population has been prepared by (i) providing nucleic acid(s) encoding a selected antibody of interest that is produced by a B cell clone or cultured plasma cells prepared as described above, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest. Thus the procedures for first preparing the recombinant host cell and then culturing it to express antibody can be performed at very different times by different people in different places (e.g., in different countries).

### Pharmaceutical Composition

The present disclosure also provides a pharmaceutical composition comprising one or more of:
(i) the antibody according to the present disclosure;
(ii) the nucleic acid encoding the antibody according to the present disclosure;
(iii) the vector comprising the nucleic acid according to the present disclosure; and/or
(iv) the cell expressing the antibody according to the present disclosure or comprising the vector according to the present disclosure
and, optionally, a pharmaceutically acceptable diluent or carrier.

In other words, the present disclosure also provides a pharmaceutical composition comprising the antibody according to the present disclosure, the nucleic acid according to the present disclosure, the vector according to the present disclosure and/or the cell according to the present disclosure.

The pharmaceutical composition may optionally also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In some embodiments, the pharmaceutically acceptable carrier, diluent and/or excipient in the pharmaceutical composition according to the present disclosure is not an active component in respect to influenza A virus infection.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions of the disclosure may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin©, for reconstitution with sterile water containing a preservative). The composition may be prepared for topical administration, e.g., as an ointment, cream or powder. The composition may be prepared for oral administration, e.g., as a tablet or capsule, as a spray, or as a syrup (optionally flavored). The composition may be prepared for pulmonary administration, e.g., as an inhaler, using a fine powder or a spray. The composition may be prepared as a suppository or pessary. The composition may be prepared for nasal, aural or ocular administration, e.g., as drops. The composition may be in kit form, designed such that a combined composition is reconstituted just prior to administration to a subject. For example, a lyophilized antibody may be provided in kit form with sterile water or a sterile buffer.

In some embodiments, the (only) active ingredient in the composition is the antibody according to the present disclosure. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions of the disclosure generally have a pH between 5.5 and 8.5, in some embodiments this may be between 6 and 8, for example about 7. The pH may be maintained by the use of a buffer. The composition may be sterile and/or pyrogen free. The composition may be isotonic with respect to humans. In some embodiments pharmaceutical compositions of the disclosure are supplied in hermetically-sealed containers.

Within the scope of the disclosure are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody may be in dry form, for reconstitution before use with an appropriate sterile liquid.

A vehicle is typically understood to be a material that is suitable for storing, transporting, and/or administering a compound, such as a pharmaceutically active compound, in particular the antibodies according to the present disclosure. For example, the vehicle may be a physiologically acceptable liquid, which is suitable for storing, transporting, and/or administering a pharmaceutically active compound, in particular the antibodies according to the present disclosure. Once formulated, the compositions of the disclosure can be administered directly to the subject. In some embodiments the compositions are adapted for administration to mammalian, e.g., human subjects.

In certain embodiments, a composition comprises a first vector comprising a first plasmid, and a second vector comprising a second plasmid, wherein the first plasmid comprises a polynucleotide encoding a heavy chain, VH, or VH+CH1, and a second plasmid comprises a polynucleotide encoding the cognate light chain, VL, or VL+CL of the antibody. In certain embodiments, a composition comprises a polynucleotide (e.g., mRNA) coupled to a suitable delivery vehicle or carrier.

Exemplary vehicles or carriers for administration to a human subject include a lipid or lipid-derived delivery vehicle, such as a liposome, solid lipid nanoparticle, oily suspension, submicron lipid emulsion, lipid microbubble, inverse lipid micelle, cochlear liposome, lipid microtubule, lipid microcylinder, or lipid nanoparticle (LNP) or a nanoscale platform (see, e.g., Li et al. Wilery Interdiscip Rev. Nanomed Nanobiotechnol. 11(2):e1530 (2019)). Principles, reagents, and techniques for designing appropriate mRNA and formulating mRNA-LNP and delivering the same are described in, for example, Pardi et al. (J Control Release 217345-351 (2015)); Thess et al. (Mol Ther 23: 1456-1464 (2015)); Thran et al. (EMBO Mol Med 9(10):1434-1448 (2017); Kose et al. (Sci. Immunol. 4 eaaw6647 (2019); and Sabnis et al. (Mol. Ther. 26:1509-1519 (2018)), which techniques, include capping, codon optimization, nucleoside modification, purification of mRNA, incorporation of the mRNA into stable lipid nanoparticles (e.g., ionizable cationic lipid/phosphatidylcholine/cholesterol/PEG-lipid; ionizable lipid:distearoyl PC:cholesterol:polyethylene glycol lipid), and subcutaneous, intramuscular, intradermal, intravenous, intraperitoneal, and intratracheal administration of the same, are incorporated herein by reference.

The pharmaceutical compositions of this disclosure may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions of the disclosure. Optionally, the pharmaceutical composition may be prepared for oral administration, e.g., as tablets, capsules and the like, for topical administration, or as injectable, e.g., as liquid solutions or suspensions. In some embodiments, the pharmaceutical composition is an injectable. Solid forms suitable for solution in, or suspension in, liquid vehicles prior to injection are also encompassed, for example the pharmaceutical composition may be in lyophilized form.

Pharmaceutical compositions may be presented in unit dose forms containing a predetermined amount of active ingredient per unit dose. As is known to those skilled in the art, the amount of active ingredient per dose will depend on the condition being treated, the route of administration and the age, weight and condition of the patient.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier.

For injection, e.g., intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is an antibody, a peptide, a nucleic acid molecule, or another pharmaceutically useful compound according to the present disclosure that is to be given to an individual, administration is usually in a “prophylactically effective amount” or a “therapeutically effective amount” (as the case may be), this being sufficient to show benefit to the individual. A therapeutically effective amount of an antibody, polynucleotide, vector, or composition of this disclosure can be an amount of the composition or molecule sufficient to result in a therapeutic effect, including improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. The actual amount administered, and rate and time-course of administration, will depend on, for example, the nature and severity of what is being treated. For injection, the pharmaceutical composition according to the present disclosure may be provided for example in a pre-filled syringe.

The pharmaceutical composition described herein may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

The pharmaceutical composition described herein may also be administered topically, especially when the target of treatment includes areas or organs readily accessible by topical application, e.g., including accessible epithelial tissue. Suitable topical formulations are readily prepared for each of these areas or organs. For topical applications, the pharmaceutical composition may be formulated in a suitable ointment, containing the pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the pharmaceutical composition can be formulated in a suitable lotion or cream. In the context of the present disclosure, suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule. In particular, the pharmaceutical composition may be provided as single-dose product. In some embodiments, the amount of the antibody in the pharmaceutical composition—in particular if provided as single-dose product—does not exceed 200 mg, for example it does not exceed 100 mg or 50 mg.

For example, the pharmaceutical composition according to the present disclosure may be administered daily, e.g., once or several times per day, e.g., once, twice, three times or four times per day, for 1, 2, 3, 4, 5, 6, 7, 8, 9, 10, 11, 12, 13, 14, 15, 16, 17, 18, 19, 20 or 21 or more days, e.g., daily for 1, 2, 3, 4, 5, 6 months. In some embodiments, the pharmaceutical composition according to the present disclosure may be administered weekly, e.g., once or twice per week, for 1, 2, 3, 4, 5, 6, 7, 8, 9, 10, 11, 12, 13, 14, 15, 16, 17, 18, 19, 20 or 21 or more weeks, e.g., weekly for 1, 2, 3, 4, 5, 6, 7, 8, 9, 10, 11, or 12 months or weekly for 2, 3, 4, or 5 years. Moreover, the pharmaceutical composition according to the present disclosure may be administered monthly, e.g., once per month or every second month for 1, 2, 3, 4, or 5 or more years. Administration may also continue for the lifetime. In some embodiments, one single administration only is also envisaged, in particular in respect to certain indications, e.g., for prophylaxis of influenza A virus infection. For example, a single administration (single dose) is administered and further doses may be administered at one or more later time points, when the titer of the antibody is insufficient or assumed to be insufficient for protection.

For a single dose, e.g., a daily, weekly or monthly dose, the amount of the antibody in the pharmaceutical composition according to the present disclosure, may not exceed 1 g or 500 mg. In some embodiments, for a single dose, the amount of the antibody in the pharmaceutical composition according to the present disclosure, may not exceed 200 mg, or 100 mg. For example, for a single dose, the amount of the antibody in the pharmaceutical composition according to the present disclosure, may not exceed 50 mg.

Pharmaceutical compositions typically include an “effective” amount of one or more antibodies of the disclosure, i.e., an amount that is sufficient to treat, ameliorate, attenuate, reduce or prevent a desired disease or condition, or to exhibit a detectable therapeutic effect. Therapeutic effects also include reduction or attenuation in pathogenic potency or physical symptoms. In some embodiments, an effective amount is an amount sufficient to result in improved clinical outcome; lessening or alleviation of symptoms associated with a disease; decreased occurrence of symptoms; improved quality of life; longer disease-free status; diminishment of extent of disease, stabilization of disease state; delay of disease progression; remission; survival; or prolonged survival in a statistically significant manner. The precise effective amount for any particular subject can depend upon, for example, their size, weight, and health, the nature and extent of the condition, rate of excretion, mode and timing of administration, and the therapeutics or combination of therapeutics selected for administration. The effective amount for a given situation is determined by routine experimentation and is within the judgment of a clinician. For purposes of the present disclosure, an effective dose may generally be from about 0.005 to about 100 mg/kg, for example from about 0.0075 to about 50 mg/kg or from about 0.01 to about 10 mg/kg. In some embodiments, the effective dose will be from about 0.02 to about 5 mg/kg, of the antibody of the present disclosure (e.g., amount of the antibody in the pharmaceutical composition) in relation to the bodyweight (e.g., in kg) of the individual to which it is administered.

In certain embodiments, following administration of therapies according to the formulations and methods of this disclosure, test subjects will exhibit about a 10% up to about a 99% reduction in one or more symptoms associated with the disease or disorder being treated as compared to placebo-treated or other suitable control subjects.

Moreover, the pharmaceutical composition according to the present disclosure may also comprise an additional active component, which may be a further antibody or a component, which is not an antibody. For example, the pharmaceutical composition may comprise one or more antivirals (which are not antibodies). Moreover, the pharmaceutical composition may also comprise one or more antibodies (which are not according to the disclosure), for example an antibody against other influenza virus antigens (other than hemagglutinin) or an antibody against another influenza virus (e.g., against an influenza B virus or against an influenza C virus). Accordingly, the pharmaceutical composition according to the present disclosure may comprise one or more of the additional active components.

The antibody according to the present disclosure can be present either in the same pharmaceutical composition as the additional active component or, alternatively, the antibody according to the present disclosure is comprised by a first pharmaceutical composition and the additional active component is comprised by a second pharmaceutical composition different from the first pharmaceutical composition. Accordingly, if more than one additional active component is envisaged, each additional active component and the antibody according to the present disclosure may be comprised in a different pharmaceutical composition. Such different pharmaceutical compositions may be administered either combined/simultaneously or at separate times or at separate locations (e.g., separate parts of the body).

The antibody according to the present disclosure and the additional active component may provide an additive therapeutic effect, such as a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

In some embodiments, a composition of the disclosure may include antibodies of the disclosure, wherein the antibodies may make up at least 50% by weight (e.g., 60%, 70%, 75%, 80%, 85%, 90%, 95%, 96%, 97%, 98%, 99% or more) of the total protein in the composition. In the composition of the disclosure, the antibodies may be in purified form.

The present disclosure also provides a method of preparing a pharmaceutical composition comprising the steps of: (i) preparing an antibody of the disclosure; and (ii) admixing the purified antibody with one or more pharmaceutically-acceptable carriers.

In other embodiments, a method of preparing a pharmaceutical composition comprises the step of: admixing an antibody with one or more pharmaceutically-acceptable carriers, wherein the antibody is a monoclonal antibody that was obtained from a transformed B cell or a cultured plasma cell of the disclosure.

As an alternative to delivering antibodies or B cells for therapeutic purposes, it is possible to deliver nucleic acid (typically DNA) that encodes the monoclonal antibody of interest derived from the B cell or the cultured plasma cells to a subject, such that the nucleic acid can be expressed in the subject in situ to provide a desired therapeutic effect. Suitable gene therapy and nucleic acid delivery vectors are known in the art.

Pharmaceutical compositions may include an antimicrobial, particularly if packaged in a multiple dose format. They may comprise detergent, e.g., a Tween (polysorbate), such as Tween 80. Detergents are generally present at low levels, e.g., less than 0.01%. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

The compositions of the disclosure may also comprise one or more immunoregulatory agents. In some embodiments, one or more of the immunoregulatory agents include(s) an adjuvant.

### Medical Treatments and Uses

In a further aspect, the present disclosure provides the use of the antibody according to the present disclosure, the nucleic acid according to the present disclosure, the vector according to the present disclosure, the cell according to the present disclosure or the pharmaceutical composition according to the present disclosure in (i) prophylaxis and/or treatment of infection with influenza A virus; or in (ii) diagnosis of infection with influenza A virus. Accordingly, the present disclosure also provides a method of reducing influenza A virus infection, or lowering the risk of influenza A virus infection, comprising: administering to a subject in need thereof, a therapeutically effective amount of the antibody according to the present disclosure, the nucleic acid according to the present disclosure, the vector according to the present disclosure, the cell according to the present disclosure or the pharmaceutical composition according to the present disclosure. Moreover, the present disclosure also provides the use of the antibody according to the present disclosure, the nucleic acid according to the present disclosure, the vector according to the present disclosure, the cell according to the present disclosure or the pharmaceutical composition according to the present disclosure in the manufacture of a medicament for prophylaxis, treatment or attenuation of influenza A virus infection.

Methods of diagnosis may include contacting an antibody with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood, such as plasma or serum. The methods of diagnosis may also include the detection of an antigen/antibody complex, in particular following the contacting of an antibody with a sample. Such a detection step is typically performed at the bench, i.e., without any contact to the human or animal body. Examples of detection methods are well-known to the person skilled in the art and include, e.g., ELISA (enzyme-linked immunosorbent assay).

Prophylaxis of infection with influenza A virus refers in particular to prophylactic settings, wherein the subject was not diagnosed with infection with influenza A virus (either no diagnosis was performed or diagnosis results were negative) and/or the subject does not show symptoms of infection with influenza A virus. Prophylaxis of infection with influenza A virus is particularly useful in subjects at greater risk of severe disease or complications when infected, such as pregnant women, children (such as children under 59 months), the elderly, individuals with chronic medical conditions (such as chronic cardiac, pulmonary, renal, metabolic, neurodevelopmental, liver or hematologic diseases) and individuals with immunosuppressive conditions (such as HIV/AIDS, receiving chemotherapy or steroids, or malignancy). Moreover, prophylaxis of infection with influenza A virus is also particularly useful in subjects at greater risk acquiring influenza A virus infection, e.g., due to increased exposure, for example subjects working or staying in public areas, in particular health care workers.

In therapeutic settings, in contrast, the subject is typically infected with influenza A virus, diagnosed with influenza A virus infection and/or showing symptoms of influenza A virus infection. Of note, the terms “treatment” and “therapy”/“therapeutic” of influenza A virus infection include (complete) cure as well as attenuation/reduction of influenza A virus infection and/or related symptoms. In some instances, a treatment or therapy/therapeutic can refer to medical management of a disease, disorder, or condition of a subject. In general, an appropriate dose or treatment regimen comprising an antibody or composition of the present disclosure is administered in an amount sufficient to elicit a therapeutic or prophylactic benefit. Therapeutic or prophylactic/preventive benefit includes improved clinical outcome; lessening or alleviation of symptoms associated with a disease; decreased occurrence of symptoms; improved quality of life; longer disease-free status; diminishment of extent of disease, stabilization of disease state; delay or prevention of disease progression; remission; survival; prolonged survival; or any combination thereof.

Accordingly, the antibody according to the present disclosure, the nucleic acid according to the present disclosure, the vector according to the present disclosure, the cell according to the present disclosure or the pharmaceutical composition according to the present disclosure may be used for treatment of influenza A virus infection in subjects diagnosed with influenza A virus infection or in subjects showing symptoms of influenza A virus infection.

The antibody according to the present disclosure, the nucleic acid according to the present disclosure, the vector according to the present disclosure, the cell according to the present disclosure or the pharmaceutical composition according to the present disclosure may also be used for prophylaxis and/or treatment of influenza A virus infection in asymptomatic subjects. Those subjects may be diagnosed or not diagnosed with influenza A virus infection.

Moreover, the antibody according to the present disclosure, the nucleic acid according to the present disclosure, the vector according to the present disclosure, the cell according to the present disclosure or the pharmaceutical composition according to the present disclosure may be used for prophylaxis and/or treatment of influenza A virus infection, wherein the antibody, the nucleic acid, the vector, the cell, or the pharmaceutical composition is administered up to three months before the first symptoms of influenza A infection occur or up to one month before the first symptoms of influenza A infection occur, such as up to two weeks the first symptoms of influenza A infection occur or up to one week before the first symptoms of influenza A infection occur. For example, the antibody according to the present disclosure, the nucleic acid according to the present disclosure, the vector according to the present disclosure, the cell according to the present disclosure or the pharmaceutical composition according to the present disclosure is used for prophylaxis and/or treatment of influenza A virus infection, wherein the antibody, the nucleic acid, the vector, the cell, or the pharmaceutical composition is administered up to three days or two days before the first symptoms of influenza A infection occur.

In general after the first administration of the antibody according to the present disclosure, the nucleic acid according to the present disclosure, the vector according to the present disclosure, the cell according to the present disclosure or the pharmaceutical composition according to the present disclosure, one or more subsequent administrations may follow, for example a single dose per day or per every second day for 1, 2, 3, 4, 5, 6, 7, 8, 9, 10, 11, 12, 13, 1, 15, 16, 17, 18, 19, 20, or 21 days. After the first administration of the antibody according to the present disclosure, the nucleic acid according to the present disclosure, the vector according to the present disclosure, the cell according to the present disclosure or the pharmaceutical composition according to the present disclosure, one or more subsequent administrations may follow, for example a single dose once or twice per week for 1, 2, 3, 4, 5, 6, 7, 8, 9, 10, 11, 12, 13, 1, 15, 16, 17, 18, 19, 20, or 21 weeks. After the first administration of the antibody according to the present disclosure, the nucleic acid according to the present disclosure, the vector according to the present disclosure, the cell according to the present disclosure or the pharmaceutical composition according to the present disclosure, one or more subsequent administrations may follow, for example a single dose every 2 or 4 weeks for 1, 2, 3, 4, 5, 6, 7, 8, 9, 10, 11, 12, 13, 1, 15, 16, 17, 18, 19, 20, or 21 weeks. After the first administration of the antibody according to the present disclosure, the nucleic acid according to the present disclosure, the vector according to the present disclosure, the cell according to the present disclosure or the pharmaceutical composition according to the present disclosure, one or more subsequent administrations may follow, for example a single dose every two or four months for 1, 2, 3, 4, 5, 6, 7, 8, 9, 10, 11, 12, 13, 1, 15, 16, 17, 18, 19, 20, or 21 months. After the first administration of the antibody according to the present disclosure, the nucleic acid according to the present disclosure, the vector according to the present disclosure, the cell according to the present disclosure or the pharmaceutical composition according to the present disclosure, one or more subsequent administrations may follow, for example a single dose once or twice per year for 1, 2, 3, 4, 5, 6, 7, 8, 9, or 10 years.

In some embodiments, the antibody according to the present disclosure, the nucleic acid according to the present disclosure, the vector according to the present disclosure, the cell according to the present disclosure or the pharmaceutical composition according to the present disclosure is administered at a (single) dose of 0.005 to 100 mg/kg bodyweight or 0.0075 to 50 mg/kg bodyweight, such as at a (single) dose of 0.01 to 10 mg/kg bodyweight or at a (single) dose of 0.05 to 5 mg/kg bodyweight. For example, the antibody according to the present disclosure, the nucleic acid according to the present disclosure, the vector according to the present disclosure, the cell according to the present disclosure or the pharmaceutical composition according to the present disclosure is administered at a (single) dose of 0.1 to 1 mg/kg bodyweight.

The antibody according to the present disclosure, the nucleic acid according to the present disclosure, the vector according to the present disclosure, the cell according to the present disclosure or the pharmaceutical composition according to the present disclosure may be administered by any number of routes such as oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes.

In some embodiments, the antibody according to the present disclosure, the nucleic acid according to the present disclosure, the vector according to the present disclosure, the cell according to the present disclosure or the pharmaceutical composition according to the present disclosure is administered prophylactically, i.e., before diagnosis of influenza A infection.

In some embodiments, the antibody of the disclosure may be administered to subjects at immediate risk of influenza A infection. An immediate risk of influenza A infection typically occurs during an influenza A epidemic. Influenza A viruses are known to circulate and cause seasonal epidemics of disease (WHO, Influenza (Seasonal) Fact sheet, Nov. 6, 2018). In temperate climates, seasonal epidemics occur mainly during winter, while in tropical regions, influenza may occur throughout the year, causing outbreaks more irregularly. For example, in the northern hemisphere, the risk of an influenza A epidemic is high during November, December, January, February and March, while in the southern hemisphere the risk of an influenza A epidemic is high during May, June, July, August and September.

### Combination Therapy

The administration of the antibody according to the present disclosure, the nucleic acid according to the present disclosure, the vector according to the present disclosure, the cell according to the present disclosure or the pharmaceutical composition according to the present disclosure in the methods and uses according to the disclosure can be carried out alone or in combination with a co-agent (also referred to as “additional active component” herein), which may be useful for preventing and/or treating influenza infection.

The disclosure encompasses the administration of the antibody according to the present disclosure, the nucleic acid according to the present disclosure, the vector according to the present disclosure, the cell according to the present disclosure or the pharmaceutical composition according to the present disclosure, wherein it is administered to a subject prior to, simultaneously with or after a co-agent or another therapeutic regimen useful for treating and/or preventing influenza. Said antibody, nucleic acid, vector, cell or pharmaceutical composition, that is administered in combination with said co-agent can be administered in the same or different composition(s) and by the same or different route(s) of administration. As used herein, expressions like “combination therapy,” “combined administration,” “administered in combination” and the like are intended to refer to a combined action of the drugs (which are to be administered “in combination”). To this end, the combined drugs are usually present at a site of action at the same time and/or at an overlapping time window. It may also be possible that the effects triggered by one of the drugs are still ongoing (even if the drug itself may not be present anymore) while the other drug is administered, such that effects of both drugs can interact. However, a drug which was administered long before another drug (e.g., more than one, two, three or more months or a year), such that it is not present anymore (or its effects are not ongoing) when the other drug is administered, is typically not considered to be administered “in combination.” For example, influenza medications administered in distinct influenza seasons are usually not administered “in combination.”

Said other therapeutic regimens or co-agents may be, for example, an antiviral. An antiviral (or “antiviral agent” or “antiviral drug”) refers to a class of medication used specifically for treating viral infections. Like antibiotics for bacteria, antivirals may be broad spectrum antivirals useful against various viruses or specific antivirals that are used for specific viruses. Unlike most antibiotics, antiviral drugs do usually not destroy their target pathogen; instead they typically inhibit their development.

Thus, in another aspect of the present disclosure, the antibody according to the present disclosure, the nucleic acid according to the present disclosure, the vector according to the present disclosure, the cell according to the present disclosure, or the pharmaceutical composition according to the present disclosure is administered in combination with (prior to, simultaneously or after) an antiviral for the (medical) uses as described herein.

In general, an antiviral may be a broad spectrum antiviral (which is useful against influenza viruses and other viruses) or an influenza virus-specific antiviral. In some embodiments, the antiviral is not an antibody. For example, the antiviral may be a small molecule drug. Examples of small molecule antivirals useful in prophylaxis and/or treatment of influenza are described in Wu X, Wu X, Sun Q, et al. Progress of small molecular inhibitors in the development of anti-influenza virus agents. Theranostics. 2017; 7(4):826-845. As described in Wu et al., 2017, the skilled artisan is familiar with various antivirals useful in prophylaxis and/or treatment of influenza. Further antivirals useful in influenza are described in Davidson S. Treating Influenza Infection, From Now and Into the Future. Front Immunol. 2018; 9:1946; and in: Koszalka P, Tilmanis D, Hurt A C. Influenza antivirals currently in late-phase clinical trial. Influenza Other Respir Viruses. 2017; 11(3):240-246.

Antivirals useful in prophylaxis and/or treatment of influenza include (i) agents targeting functional proteins of the influenza virus itself and (ii) agents targeting host cells, e.g., the epithelium.

Host cell targeting agents include the thiazolide class of broad-spectrum antivirals, sialidase fusion proteins, type III interferons, Bcl-2 (B cell lymphoma 2) inhibitors, protease inhibitors, V-ATPase inhibitors and antioxidants. Examples of the thiazolide class of broad-spectrum antivirals include nitazoxanide (NTZ), which is rapidly deacetylated in the blood to the active metabolic form tizoxanide (TIZ), and second generation thiazolide compounds, which are structurally related to NTZ, such as RM5061. Fludase (DAS181) is an example for sialidase fusion proteins. Type III IFNs include, for example, IFNλ. Non-limiting examples of Bcl-2 inhibitors include ABT-737, ABT-263, ABT-199, WEHI-539 and A-1331852 (Davidson S. Treating Influenza Infection, From Now and Into the Future. Front Immunol. 2018; 9:1946). Examples of protease inhibitors include nafamostat, Leupeptin, epsilon-aminocapronic acid, Camostat and Aprotinin. V-ATPase inhibitors include NorakinR, ParkopanR, AntiparkinR and AkinetonR. An example of an antioxidant is alpha-tocopherol.

In some embodiments, the antiviral is an agent targeting a functional protein of the influenza virus itself. For example, the antiviral may target a functional protein of the influenza virus, which is not hemagglutinin. In general, antivirals targeting a functional protein of the influenza virus include entry inhibitors, hemagglutinin inhibitors, neuraminidase inhibitors, influenza polymerase inhibitors (RNA-dependent RNA polymerase (RdRp) inhibitors), nucleocapsid protein inhibitors, M2 ion channel inhibitors and arbidol hydrochloride. Non-limiting examples of entry inhibitors include triterpenoids derivatives, such as glycyrrhizic acid (glycyrrhizin) and glycyrrhetinic acid; saponins; uralsaponins M-Y (such as uralsaponins M); dextran sulphate (DS); silymarin; curcumin; and lysosomotropic agents, such as Concanamycin A, Bafilomycin A1, and Chloroquine. Non-limiting examples of hemagglutinin inhibitors include BMY-27709; stachyflin; natural products, such as Gossypol, Rutin, Quercetin, Xylopine, and Theaflavins; trivalent glycopeptide mimetics, such as compound 1 described in Wu X, Wu X, Sun Q, et al. Progress of small molecular inhibitors in the development of anti-influenza virus agents. Theranostics. 2017; 7(4):826-845; podocarpic acid derivatives, such as compound 2 described in Wu X, Wu X, Sun Q, et al. Progress of small molecular inhibitors in the development of anti-influenza virus agents. Theranostics. 2017; 7(4):826-845; natural product pentacyclic triterpenoids, such as compound 3 described in Wu X, Wu X, Sun Q, et al. Progress of small molecular inhibitors in the development of anti-influenza virus agents. Theranostics. 2017; 7(4):826-845; and prenylated indole diketopiperazine alkaloids, such as Neoechinulin B. Non-limiting examples of nucelocapsid protein inhibitors include nucleozin, Cycloheximide, Naproxen and Ingavirin. Non-limiting examples of M2 ion channel inhibitors include the approved M2 inhibitors Amantadine and Rimantadine and derivatives thereof, as well as non-adamantane derivatives, such as Spermine, Spermidine, Spiropiperidine and pinanamine derivatives.

In some embodiments, the antiviral is selected from neuraminidase (NA) inhibitors and influenza polymerase inhibitors (RNA-dependent RNA polymerase (RdRp) inhibitors). Non-limiting examples of neuraminidase (NA) inhibitors include zanamivir; oseltamivir; peramivir; laninamivir; derivatives thereof such as compounds 4-10 described in Wu X, Wu X, Sun Q, et al. Progress of small molecular inhibitors in the development of anti-influenza virus agents. Theranostics. 2017; 7(4):826-845, and dimeric zanamivir conjugates (e.g., as described in Wu X, Wu X, Sun Q, et al. Progress of small molecular inhibitors in the development of anti-influenza virus agents. Theranostics. 2017; 7(4):826-845); benzoic acid derivatives (e.g., as described in Wu X, Wu X, Sun Q, et al. Progress of small molecular inhibitors in the development of anti-influenza virus agents. Theranostics. 2017; 7(4):826-845; such as compounds 11-14); pyrrolidine derivatives (e.g., as described in Wu X, Wu X, Sun Q, et al. Progress of small molecular inhibitors in the development of anti-influenza virus agents. Theranostics. 2017; 7(4):826-845; such as compounds 15-18); ginkgetin-sialic acid conjugates; flavanones and flavonoids isoscutellarein and its derivatives (e.g., as described in Wu X, Wu X, Sun Q, et al. Progress of small molecular inhibitors in the development of anti-influenza virus agents. Theranostics. 2017; 7(4):826-845); AV5080; and N-substituted oseltamivir analogues (e.g., as described in Wu X, Wu X, Sun Q, et al. Progress of small molecular inhibitors in the development of anti-influenza virus agents. Theranostics. 2017; 7(4):826-845). Non-limiting examples of influenza polymerase inhibitors (RNA-dependent RNA polymerase (RdRp)) inhibitors include RdRp disrupting compounds, such as those described in Wu X, Wu X, Sun Q, et al. Progress of small molecular inhibitors in the development of anti-influenza virus agents. Theranostics. 2017; 7(4):826-845; PB2 cap-binding inhibitors, such as JNJ63623872 (VX-787); cap-dependent endonuclease inhibitors, such as baloxavir marboxil (5-033188); PA endonuclease inhibitors, such as AL-794, EGCG and its aliphatic analogues, N-hydroxamic acids and N-hydroxyimides, flutimide and its aromatic analogues, tetramic acid derivatives, L-742,001, ANA-0, polyphenolic catechins, phenethyl-phenylphthalimide analogues, macrocyclic bisbibenzyls, pyrimidinoles, fullerenes, hydroxyquinolinones, hydroxypyridinones, hydroxypyridazinones, trihydroxy-phenyl-bearing compounds, 2-hydroxy-benzamides, hydroxy-pyrimidinones, β-diketo acid and its bioisosteric compounds, thiosemicarbazones, bisdihydroxyindole-carboxamides, and pyrido-piperazinediones (Endo-1); and nucleoside and nucleobase analogue inhibitors, such as ribavirin, favipiravir (T-705), 2′-Deoxy-2′-fluoroguanosine (2′-FdG), 2′-substituted carba-nucleoside analogues, 6-methyl-7-substituted-7-deaza purine nucleoside analogues, and 2′-deoxy-2′-fluorocytidine (2′-FdC). For example, the antiviral may be zanamivir, oseltamivir or baloxavir.

Thus, the pharmaceutical composition according to the present disclosure may comprise one or more of the additional active components. The antibody according to the present disclosure can be present in the same pharmaceutical composition as the additional active component (co-agent). Alternatively, the antibody according to the present disclosure and the additional active component (co-agent) are comprised in distinct pharmaceutical compositions (e.g., not in the same composition). Accordingly, if more than one additional active component (co-agent) is envisaged, each additional active component (co-agent) and the antibody according to the present disclosure may be comprised by a different pharmaceutical composition. Such different pharmaceutical compositions may be administered either combined/simultaneously or at separate times and/or by separate routes of administration.

The antibody according to the present disclosure and the additional active component (co-agent) may provide an additive or a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

Accordingly, the present disclosure also provides a combination of (i) the antibody of the disclosure as described herein, and (ii) an antiviral agent as described above.

### EXAMPLES

In the following, particular examples illustrating various embodiments and aspects of the disclosure are presented. However, the present disclosure shall not to be limited in scope by the specific embodiments described herein. The following preparations and examples are given to enable those skilled in the art to more clearly understand and to practice the present disclosure. The present disclosure, however, is not limited in scope by the exemplified embodiments, which are intended as illustrations of single aspects of the disclosure only, and methods which are functionally equivalent are within the scope of the disclosure. Indeed, various modifications of the disclosure in addition to those described herein will become readily apparent to those skilled in the art from the foregoing description, accompanying figures and the examples below. All such modifications fall within the scope of the appended claims.

### Example 1

### Binding of Antibodies According to the Present Disclosure to Hemagglutinin

An antibody according to the present disclosure, which comprises (i) the CDR sequences as set forth in SEQ ID NOs 1-6 and (ii) the three mutations G236A, A330L and I332E in the heavy chain constant regions, was designed and produced. More specifically, the antibody comprises (i) the heavy chain variable region (VH) sequence as set forth in SEQ ID NO: 7 and the light chain variable region (VL) sequence as set forth in SEQ ID NO: 8; and (ii) the three mutations G236A, A330L and I332E in the heavy chain constant regions. Even more specifically, the antibody comprises a heavy chain having an amino acid sequence as set forth in SEQ ID NO: 9 and a light chain having an amino acid sequence as set forth in SEQ ID NO: 10. This antibody is referred to herein as “FluAB_GAALIE.” In particular, the constant regions of antibody “FluAB_GAALIE” do not comprise any other mutations (other than G236A, A330L and I332E).

In addition, another antibody according to the present disclosure was designed and produced, which differs from antibody “FluAB_GAALIE” only in that it also comprises, in its heavy chain constant region, the two mutations M428L and N434S in addition to the three mutations G236A, A330L and I332E. Accordingly, this antibody has a heavy chain having an amino acid sequence as set forth in SEQ ID NO: 13 and a light chain having an amino acid sequence as set forth in SEQ ID NO: 10, and is referred to herein as “FluAB_GAALIE+MLNS.”

For comparison, antibody “FluAB_wt” was used, which differs from antibody “FluAB_GAALIE” only in that it does not contain the three mutations G236A, A330L and I332E in the heavy chain constant regions. Accordingly, comparative antibody “FluAB_wt” comprises a heavy chain having an amino acid sequence as set forth in SEQ ID NO: 11 and a light chain having an amino acid sequence as set forth in SEQ ID NO: 10.

In addition, also antibody “FluAB_MLNS” was used for comparison, which differs from “FluAB_wt” only in that it comprises the two mutations M428L and N434S. As “FluAB_wt,” “FluAB_MLNS” does not comprise the three mutations G236A, A330L and I332E in the heavy chain constant regions. Accordingly, comparative antibody “FluAB_MLNS” comprises a heavy chain having an amino acid sequence as set forth in SEQ ID NO: 12 and a light chain having an amino acid sequence as set forth in SEQ ID NO: 10.

The antibodies were tested for their ability to bind to influenza hemagglutinin (HA). To this end, the antibodies of the disclosure “FluAB_GAALIE” and “FluAB_GAALIE+MLNS” and comparative antibodies “FluAB_wt” and “FluAB_MLNS” were tested in an in vitro ELISA assay. Briefly, half-area (0.16 cm²/well) 96-well plates were coated with 25 μl/well HA antigens (A/California/07/09) at 2 μg/ml and incubated over night at 4° C. Plates were washed once with 220 μl/well PBS-T using an automated washer. Blocking solution (100 μl/well) was added and plates further incubated for 2 hours at room temperature (RT). Blocking solution was removed and 25 μl/well of mAb serial 1:3 dilutions (concentration range from 2 to 0.1 μg/ml; performed in duplicate) in blocking buffer were dispensed and plates were incubated 1 hour at room temperature (RT). Plates were then washed 4 times with PBS-T (220 μl/well). The AP secondary antibody reagent (0.16 μg/ml, in blocking buffer) was added and further incubated for 45 min at RT. After 4 washes with PBS-T, 40 μl/well of p-NPP ELISA substrate solution was dispensed in each well and plates were developed for 15 min at RT. EC50 values were calculated using non-linear regression of log (agonist) versus response in Graph Pad Prism.

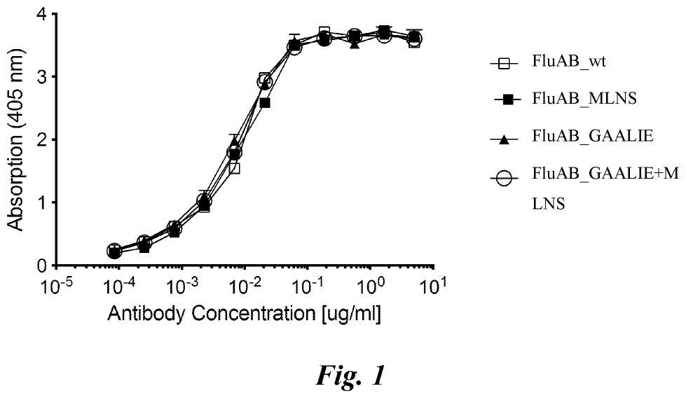

Results are shown in FIG. 1. All tested antibodies, “FluAB_GAALIE,” “FluAB_GAALIE+MLNS,” “FluAB wt” and “FluAB_MLNS,” show comparable binding to the HA antigen. Accordingly, the Fc modifications do not impair the antibodies' binding capacity.

### Example 2

### Neutralization of Influenza Virus by Antibodies According to the Present Disclosure

Next, the ability of antibodies of the disclosure “FluAB_GAALIE” and “FluAB_GAALIE+MLNS” and comparative antibodies “FluAB_wt” and “FluAB_MLNS” to neutralize influenza virus was tested in an in vitro neutralization assay. To this end, Madin-Darby Canine Kidney (MCDK) cells were seeded at 30,000 cells/well into 96-well plates (flat bottom). Cells were cultured at 37° C. 5% C02 overnight. Twenty-four hours later, 2× mAbs solutions in 60 μl Infection Medium were prepared using 1:2 7-point serial dilutions in quadruplicate (starting point: 100 μg/ml final concentration in the well). Virus solution of influenza viruses A/California/07/09 (H1N1) and A/Aichi/2/68 (H3N2) were prepared in Infection Medium at concentrations of 120 TCID₅₀ in 60 μl, further diluted either 1:1 in MEM or mixed 1:1 with mAb dilutions and incubated 1 hour at 33° C. Cells were washed 2 times using 200 μl/well MEM without supplements, followed by the addition of mAb/virus mix in Infection Medium (100 TCID₅₀/well) and incubated 4 hours at 33° C. 5% C02. After the addition of 100 μl/well of Infection Medium, cells were further incubated for 72 hours at 33° C. 5% C02. To confirm the actual TCID₅₀ of the input virus used in the neutralization assay for each viral strain, a TCID₅₀ viral titer quantification was carried out in parallel. For this assay, the virus solution prepared as described above was mixed 1:1 with Infection medium (replacing mAb solution) and 2-fold serially diluted in Infection Medium. One-hundred ml were added to quadruplicate wells and incubated in parallel to the microneutralization assay at 33° C. 5% C02 for one hour prior to addition of 100 μl to MDCK cells. Resulting titers were determined by TCID₅₀ using the “Karber method” (Spearman-Karber: Hamilton M A, Russo R C, Thurston R V (1977) Trimmed Spearman-Karber method for estimated median lethal concentration in toxicity. Environmental Science and Technology 11: 714-719) with positive samples defined as those showing >10 standard deviations above the mean value of the cells alone. On day 3 after infection 20 μM MuNANA (4-MUNANA (2_-(4-Methylumbelliferyl)-α-D-N-acetylneuraminic acid sodium salt hydrate (Sigma-Aldrich) #69587) solution was prepared in MuNANA buffer and 50 μl/well was dispensed into black 96-well plates. Fifty μl of either neutralization or virus-alone titration supernatant were transferred to the plates and incubated 60 min at 37° C. The reaction was then stopped with 100 μl/well 0.2 M glycine/50% EtOH, pH 10.7. Fluorescence was quantified at 460 nm with a fluorimeter (Promega). The percent of virus neutralization was calculated according to the formula:

$$1 - ( \frac{f x - f min}{f max} ) * 100 ,$$

where fx=sample fluorescence signal (cells+virus+mAb); fmin=minimal fluorescence signal (cells alone, no virus); fmax=maximal fluorescence signal (cells+virus only).

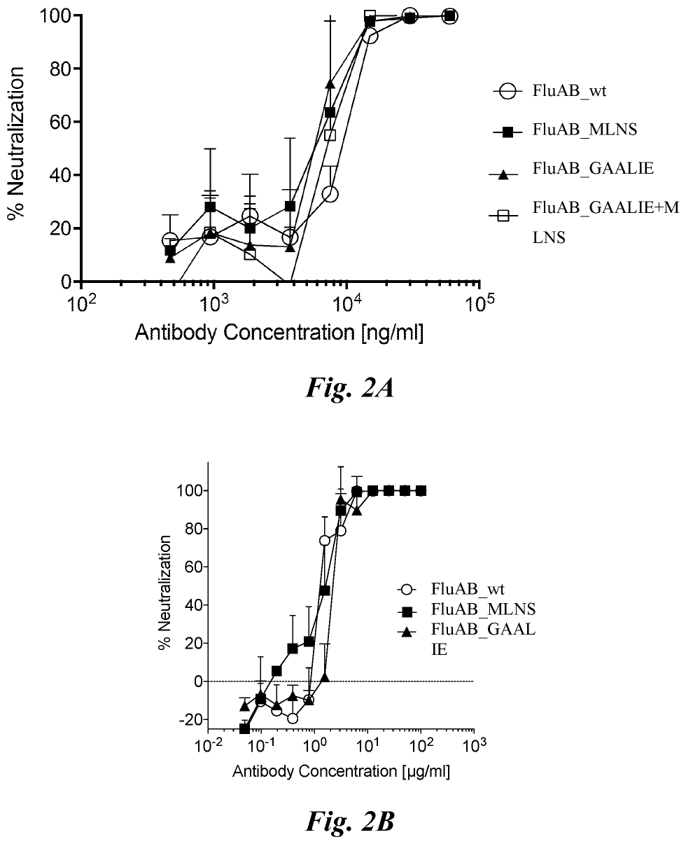

Results are shown in FIG. 2 with neutralization of (A) H1N1 strain A/California/07/09 and (B) H3N2 strain A/Aichi/2/68. All tested antibodies, “FluAB_GAALIE,” “FluAB_GAALIE+MLNS,” “FluAB_wt” and “FluAB_MLNS,” were able to neutralize both influenza viruses with comparable activity. Accordingly, the Fc modifications do not impair the antibodies' neutralization ability.

### Example 3

### Binding of the Antibodies to Human Fcγ Receptors

For an antibody, Fc-dependent mechanisms of action mediated by the interaction of the antibody's Fc region with Fc gamma receptors (FcγRs; FcgammaRs; FcgRs) on immune cells, or with complement, can make important contributions to the antibody's overall potency. FcγR-dependent mechanisms can be assessed in vitro by measuring binding to FcγRs as well as in antibody-dependent cytotoxicity assays designed to demonstrate antibody dependent cellular cytotoxicity (ADCC), complement dependent cytotoxicity (CDC), or antibody dependent cellular phagocytosis (ADCP) (Dilillo, D. J., Tan, G. S., Palese, P., & Ravetch, J. V. (2014). Broadly neutralizing hemagglutinin stalk-specific antibodies require FcγR interactions for protection against influenza virus in vivo. Nat Med, 20(2), 143-151; Henry Dunand, C. J., Leon, P. E., Huang, M., Choi, A., Chromikova, V., Ho, I. Y., et al. (2016). Both Neutralizing and Non-Neutralizing Human H7N9 Influenza Vaccine-Induced Monoclonal Antibodies Confer Protection. Cell Host Microbe, 19(6), 800-813; Kallewaard, N. L., Corti, D., Collins, P. J., Neu, U., McAuliffe, J. M., Benjamin, E., et al. (2016). Structure and Function Analysis of an Antibody Recognizing All Influenza A Subtypes. Cell, 166(3), 596-608).

Antibodies of the disclosure “FluAB_GAALIE” and “FluAB_GAALIE+MLNS” and comparative antibodies “FluAB wt” and “FluAB_MLNS” were compared side by side for their ability to bind to the full set of human FcγRs (FcγRIIIa V158 and F158 alleles, FcγRIIa H131 and R131 alleles and FeγRIIb) by biolayer interferometry (BLI). Briefly, His-tagged human FcgRs (FcgRIIa allele H131, FcgRIIa allele R131, FcgRIIIa allele F158, FcgRIIIa allele V158, and FcgRIIb) at 2 μg/ml were captured onto anti-penta-His sensors for 6 minutes. FcgRs-loaded sensors were then exposed for 4 minutes to a solution of kinetics buffer (pH 7.1) containing 2 μg/ml of each mAb in the presence 1 μg/ml of affiniPure F(ab′)₂ Fragment Goat Anti-Human IgG, F(ab′)₂ fragment specific (to cross-link antibodies through the Fab fragment), followed by a dissociation step in the same buffer for additional 4 minutes (right part of the plot). Association and dissociation profiles were measured in real time as change in the interference pattern using an Octet RED96 (FortéBio).

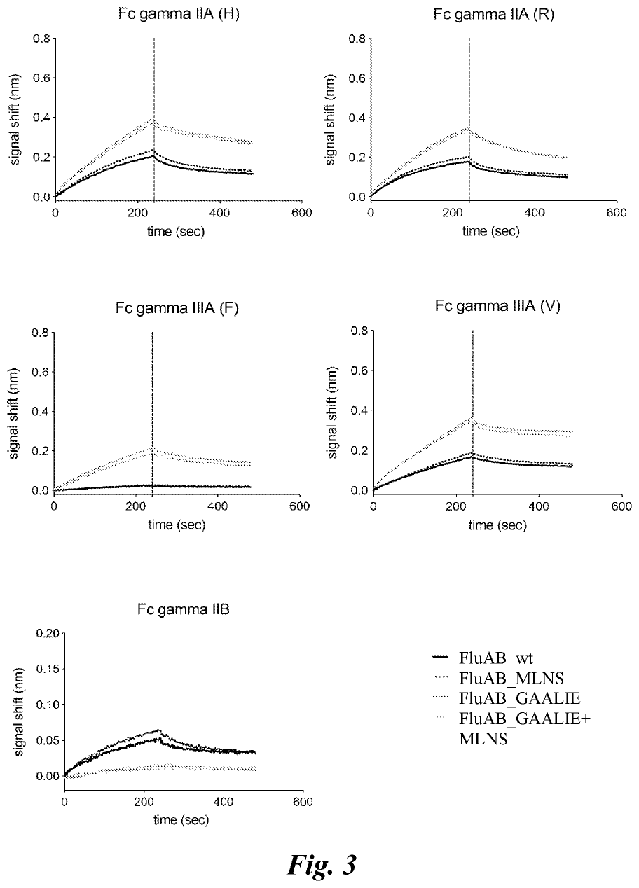

Results are shown in FIG. 3. No change in engagement of “FluAB_MLNS” with FcgRs was observed in comparison to “FluAB_wt.” Accordingly, the “MLNS” mutation did not influence engagement with FcgRs. In contrast, the antibodies of the present disclosure, “FluAB_GAALIE” and “FluAB_GAALIE+MLNS” showed enhanced binding to FcγRIIIa V158 and F158 alleles, and to FcγRIIa H131 and R131 alleles and reduced binding to FcγRIIb. These results show that antibodies of the present disclosure exhibit enhanced binding to FcγRIIa and FcγRIIIa, which is not dependent on the FcγR-alleles. In contrast, binding of antibodies of the present disclosure to the inhibitory FcγRIIb is reduced.

### Example 4

### Binding of the Antibodies to Complement (C1Q)

Next, binding of the antibody of the disclosure “FluAB_GAALIE” and of the comparative antibody “FluAB_wt” to human complement protein C1q was assessed by biolayer interferometry (BLI). Briefly, anti-human Fab (CH1-specific) sensors were used to capture through the Fab fragment the full IgG1 of mAbs at 10 μg/ml for 10 minutes. IgG-loaded sensors were then exposed for 4 minutes to a solution of kinetics buffer (pH 7.1) containing 3 μg/ml of purified human C1q (left part of the plot), followed by a dissociation step in the same buffer for additional 4 minutes (right part of the plot). Association and dissociation profiles were measured in real time as change in the interference pattern using an Octet RED96 (FortéBio).

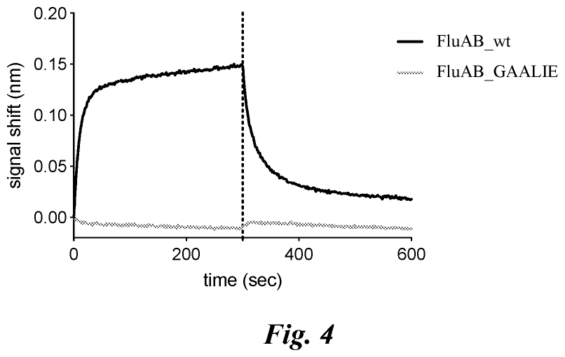

Results are shown in FIG. 4. While the comparative antibody “FluAB_wt” binds to human complement protein C1q, the ability to bind to complement is abolished in the antibody of the disclosure “FluAB_GAALIE.”

### Example 5

### Effects of Antibodies on Activation of Fcγ Receptors (ADCP and ADCC)

Antibody-dependent cellular cytotoxicity (ADCC) and antibody-dependent cell-mediated phagocytosis (ADCP) are important mechanisms of action of therapeutic antibodies designed to recognize and neutralize viruses. While ADCC is primarily mediated by FcγRIIIa expressed on NK cells, FcγRIIa is involved in ADCP.

To investigate the activation of FcγRs by antibodies of the disclosure and whether antibodies of the disclosure promote ADCP and ADCC, cell-based reporter bioassays were used. These assays utilize Jurkat cells engineered with a NFAT-mediated luciferase reporter to reflect activation of human FcγRs.

For assessing FcγRIIIa-mediated ADCC antibodies of the disclosure “FluAB_GAALIE” and “FluAB_GAALIE+MLNS” and comparative antibodies “FluAB_wt” and “FluAB_MLNS” were serially diluted 3-fold in ADCC Assay buffer from 0.3 μg/ml to 0.005 μg/ml. Target cells (A549-H1) were added in a white flat bottom 96-well plate at 12.5×10³ cells/well in 25 μl, then serially diluted antibodies were added to each well (25 μl per well), and the antibody/cell mixture was incubated for 10 minutes at room temperature. Effector cells for the ADCC Bioassay are thawed and added at a cell density of 7.5×10⁴/well in 25 μl, yielding an effector to target ratio of 6:1. Control wells were also included that were used to measure antibody-independent activation (containing target cells and effector cells but no antibody) and spontaneous luminescence of the plate (wells containing the ADCC Assay buffer only). Plates were incubated for 20 hours at 37° C. with 5% C02. Activation of human FcγRIIIa (V158 or F158 variants) in this bioassay results in the NFAT-mediated expression of the luciferase reporter gene. Luminescence is therefore measured with a luminometer using the Bio-Glo-TM Luciferase Assay Reagent according to the manufacturer's instructions. The data (i.e., specific FcgRIIIa activation) are expressed as the average of relative luminescence units (RLU) over the background by applying the following formula: (RLU at concentration x of mAbs−RLU of background).

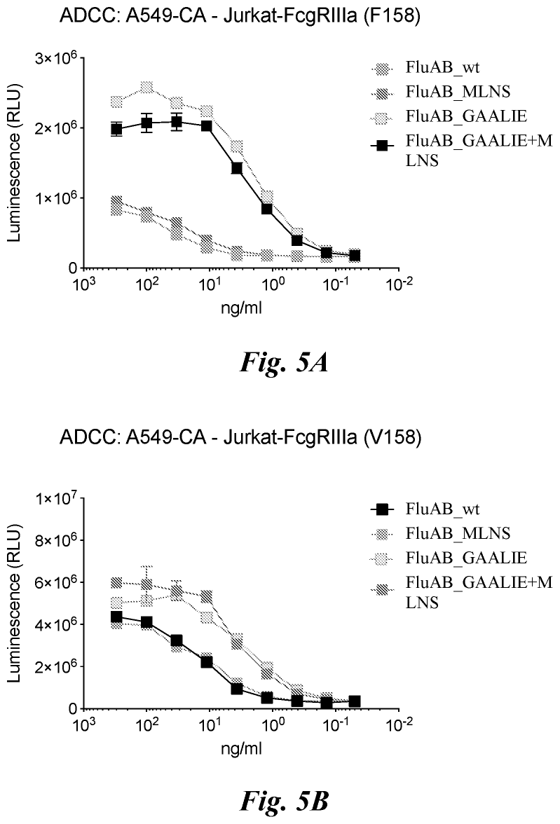

Results are shown in FIG. 5. While comparative antibodies “FluAB_wt” and “FluAB_MLNS” promote similar ADCC (similar functional activation of FcγRIIIa, independent from the allele), antibodies of the disclosure “FluAB_GAALIE” and “FluAB_GAALIE+MLNS” show enhanced activation of FcγRIIIa (both alleles), i.e., enhanced ADCC.

For assessing FcγRIIIa-mediated ADCP antibodies of the disclosure “FluAB_GAALIE” and “FluAB_GAALIE+MLNS” and comparative antibodies “FluAB_wt” and “FluAB_MLNS” were serially diluted 4-fold in ADCP Assay buffer from 5.0 μg/ml to 0.008 μg/ml. Target cells (A549-H1) were added in a white flat bottom 96-well plate at 10.0×10³ cells/well in 25 μl, then serially diluted antibodies were added to each well (25 μl per well), and the antibody/cell mixture was incubated for 10 minutes at room temperature. Effector cells for the ADCP Bioassay are thawed and added at a cell density of 50.0×10⁴/well in 25 μl, yielding an effector to target ratio of 5:1. Control wells were also included that were used to measure antibody-independent activation (containing target cells and effector cells but no antibody) and spontaneous luminescence of the plate (wells containing the ADCP Assay buffer only). Plates were incubated for 20 hours at 37° C. with 5% C02. Activation of human FcγRIIa (H131 variants) in this bioassay results in the NFAT-mediated expression of the luciferase reporter gene. Luminescence is therefore measured with a luminometer using the Bio-Glo-TM Luciferase Assay Reagent according to the manufacturer's instructions. The data (i.e., specific FcgRIIa activation) are expressed as the average of relative luminescence units (RLU) over the background by applying the following formula: (RLU at concentration x of mAbs−RLU of background).

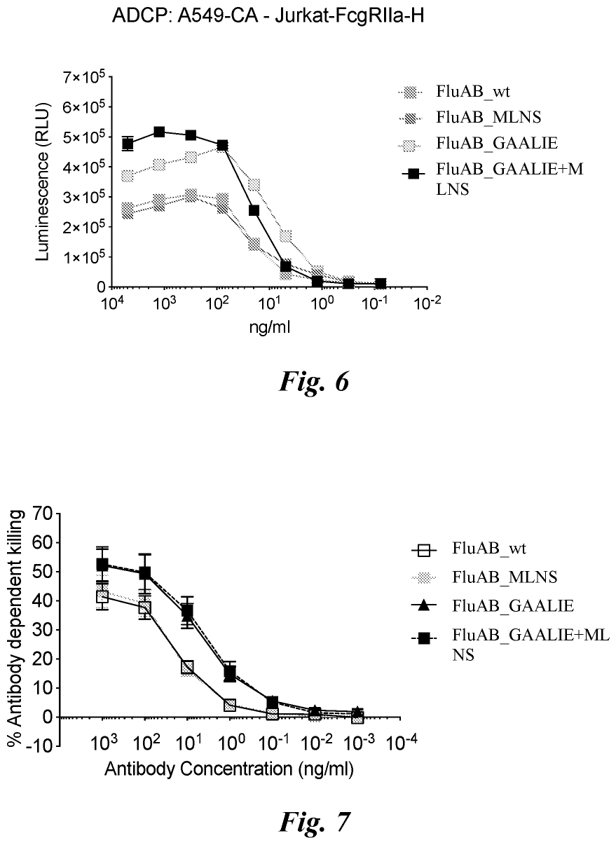

Results are shown in FIG. 6. Similar to FcγRIIIa-mediated ADCC, comparative antibodies “FluAB_wt” and “FluAB_MLNS” promote similar ADCP (similar functional activation of FcγRIIa). In comparison, antibodies of the disclosure “FluAB_GAALIE” and “FluAB_GAALIE+MLNS” show enhanced activation of FcγRIIa, i.e., enhanced ADCP.

### Example 6

### Effects of Antibodies on ADCC

Antibody-dependent cellular cytotoxicity (ADCC) activity was also measured using natural killer (NK) cells isolated from human peripheral blood mononuclear cells of one donor previously genotyped for expressing homozygous low (F158) affinity FcγRIIIa. Isolated NK cells were used to measure the killing of A549-H1 cells upon exposure to antibodies of the disclosure “FluAB_GAALIE” and “FluAB_GAALIE+MLNS” and comparative antibodies “FluAB wt” and “FluAB_MLNS.”

To this end, human NK cells were prepared from whole blood. NK cells were freshly isolated from whole EDTA blood using the MACSxpress NK isolation Kit following the manufacturer instruction. Briefly, anticoagulated blood is mixed in a 50 ml tube with 15 ml of the NK isolation cocktail and incubated for 5 minutes at room temperature using a rotator at approximately 12 round per minute. The tube is then placed in the magnetic field of the MACSxpress Separator for 15 minutes. The magnetically labeled cells will adhere to the wall of the tube while the aggregated erythrocytes sediment to the bottom. The target NK cells are then collected from the supernatant while the tube is still inside the MACSxpress Separator. NK cells are centrifuged, treated with distilled water to remove residual erythrocytes, centrifuged again and finally re-suspended in AIM-V medium.

Antibodies of the disclosure “FluAB_GAALIE” and “FluAB_GAALIE+MLNS” and comparative antibodies “FluAB_wt” and “FluAB_MLNS” were serially diluted 10-fold in AIM-V medium from 1 μg/ml to 0.001 μg/ml. Target cells (A549-H1) were added in a round bottom 384-well plate at 7.5×10³ cells/well in 23 μl, then serially diluted antibodies were added to each well (23 μl per well), and the antibody/cell mixture was incubated for 10 minutes at room temperature. After incubation, human NK cells were added at a cell density of 4.5×10⁴/well in 23 μl, yielding an effector to target ratio of 6:1. Control wells were also included that were used to measure maximal lysis (containing target cells with 23 μl of 3% Triton x-100) and spontaneous lysis (containing target cells and effector cells without antibody). Plates were incubated for 4 hours at 37° C. with 5% C02. Cell death was determined by measuring lactate dehydrogenase (LDH) release using a LDH detection kit according to the manufacturer's instructions. In brief, plates were centrifuged for 4 minutes at 400×g, and 35 μl of supernatant was transferred to a flat 384-well plate. LDH reagent was prepared and 35 μl were added to each well. Using a kinetic protocol the absorbance at 490 nm and 650 nm was measured once every 2 minutes for 8 minutes. The percent specific lysis was determined by applying the following formula: (specific release−spontaneous release)/(maximum release −spontaneous release)×100.

Results are shown in FIG. 7. The data show a similar dose-dependent cell killing in the presence of comparative antibodies “FluAB_wt” and “FluAB_MLNS.” In comparison, antibodies of the disclosure “FluAB_GAALIE” and “FluAB_GAALIE+MLNS” showed an increased dose-dependent cell killing.

### Example 7

### The “GAALIE”-Mutation in Antibodies of the Disclosure does not Compromise the Effects of a Half-Life Increasing Fc Modification In Vitro

The effects of the “GAALIE” mutation of the antibodies of the present disclosure on an additional, half-life extending Fc mutation, namely “MLNS” (Zalevsky J, Chamberlain A K, Horton H M, et al. Enhanced antibody half-life improves in vivo activity. Nat Biotechnol. 2010; 28(2):157-159), was investigated. To this end, binding of test antibodies to human FcRn in acidic milieu was assessed by biolayer interferometry (BLI). Increased FcRn binding in the acidic milieu of endosomal compartments may increase re-shuttling of antibodies to the circulation, thereby resulting into increased half-life in vivo.

Briefly, binding of antibodies of the disclosure “FluAB_GAALIE” and “FluAB_GAALIE+MLNS” and comparative antibodies “FluAB_wt” and “FluAB_MLNS” to human FcRn was measured on an Octet RED96 instrument (biolayer interferometry, BLI, ForteBio). Biosensors coated with anti-human Fab-CH1 were pre-hydrated in kinetic buffer for 10 min at RT. Then, antibodies FluAB_GAALIE, FluAB_GAALIE+MLNS, FluAB_wt and FluAB_MLNS, respectively, were loaded at 1 μg/ml in kinetics buffer at pH 7.4 for 30 minutes onto the Biosensors. The baseline was measured in kinetics buffer at pH=6.0 for 4 minutes. Antibody-loaded sensors were then exposed for 7 minutes to a solution of human FcRn at 1 μg/ml in kinetics buffer at pH=6.0 to measure association of FcRn-mAb in different milieus (on rate). Dissociation was then measured in kinetics buffer at the same pH for additional 5 minutes (off rate). All steps were performed while stirring at 1000 rpm at 30° C. Association and dissociation profiles were measured in real time as change in the interference patterns.

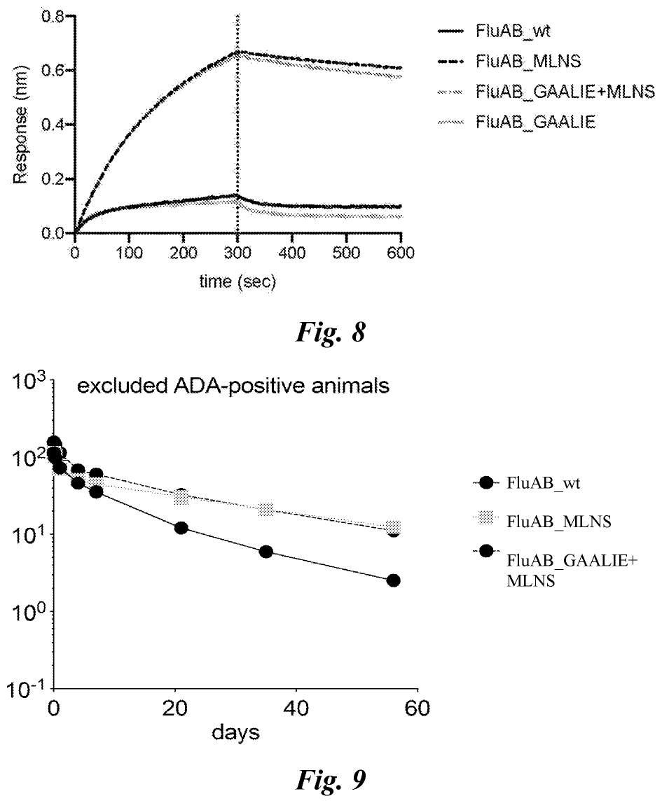

Results are shown in FIG. 8. In line with the presence of the “MLNS”-Fc, antibodies “FluAB_MLNS” and “FluAB_GAALIE+MLNS” showed an increased binding affinity to human FcRn compared to “FluAB wt” and “FluAB_GAALIE” at acidic pH 6.0. This increased binding in an acidic environment mimics the milieu of endosomal compartments and is expected to mediate shuttling of the antibodies to the extracellular compartments (Dickinson B L, Badizadegan K, Wu Z, Ahouse J C, Zhu X, Simister N E, Blumberg R S, Lencer W I. Bidirectional FcRn-dependent IgG transport in a polarized human intestinal epithelial cell line. J Clin Invest. 1999 October; 104(7):903-11), thus rescuing these mAbs from lysosomal degradation, thereby increasing their half-life. In contrast, antibodies “FluAB wt” and “FluAB GAALIE,” which do not comprise a half-life extending Fc mutation, bound human FcRn at acidic pH 6.0 only weakly. Of note, the presence of the “GAALIE” mutation did not alter the enhanced binding to huFcRn induced by the “MLNS”-mutation.

### Example 8

### The “GAALIE”-Mutation in Antibodies of the Disclosure does not Compromise the Half-Life Increasing Effects of a Respective Fc Modification In Vivo

To investigate whether the “GAALIE”-mutation of antibodies of the disclosure affects an additional, half-life extending Fc mutation in vivo, antibody “FluAB_GAALIE+MLNS” (comprising “GAALIE” and “MLNS” mutations), antibody “FluAB_MLNS” (comprising the “MLNS” mutation only) and antibody “FluAB_wt” (without Fc modification) were compared.

The antibodies were administered intravenously at a single dose of 5 mg/kg via a 60-minutes infusion into female cynomolgus monkeys (Macaca fascicularis). Blood was collected and processed to plasma for pharmacokinetic and immunogenicity testing at 1 and 6 hours (h), as well as 1, 4, 7, 21, 35, and 56 days post-dose. At days 86 and 113 post-dose, blood of two animals from the groups receiving FluAB_MLNS or FluAB_GAALIE+MLNS was collected and tested to assess the antibodies' in vivo integrity.

The concentration of antibodies FluAB_GAALIE+MLNS, FluAB_MLNS, and FluAB_wt in cynomolgus monkey plasma was determined by ELISA. Briefly, an ELISA plate was coated with influenza A virus (IAV) H1N1 (A/California/07/2009) hemagglutinin (HA) protein antigen (IAV-HA) with HisTag (SinoBiologicals #11085-V08H) at 2 μg/ml in PBS overnight at 4° C. Samples and standards were then added to the washed and blocked plate and incubated for 1 h at RT. Detection was achieved by addition of goat anti human-IgG HRP conjugate followed by SureBlue TMB Substrate to develop the plate and HCl to stop the reaction. Absorbance (optical density, OD) was measured at 450 nm using a spectrophotometer (quantitative range: 1 to 4.5 ng/ml).

To determine the concentration of the antibodies in cynomolgus plasma, OD values from ELISA data were plotted vs. concentration in the Gen5 software (BioTek). A non-linear curve fit was applied using a variable slope model, four parameters and the equation: Y=(A−D)/(1+(X/C)\hat{ }B)+D). The OD values of the sample dilutions that fell within the predictable assay range of the standard curve—as determined in setup experiment by quality control samples in the upper, medium or lower range of the curve—were interpolated to quantify the samples. Plasma concentration of the antibodies were then determined considering the final dilution of the sample. If more than one value of the sample dilutions fell within the linear range of the standard curve, an average of these values was used. Graphing and statistical analyses (linear regression or outlier analysis) were performed using Prism 7.0 software (GraphPad, La Jolla, CA, USA). Animals developing anti-drug antibody (ADA) response were excluded.

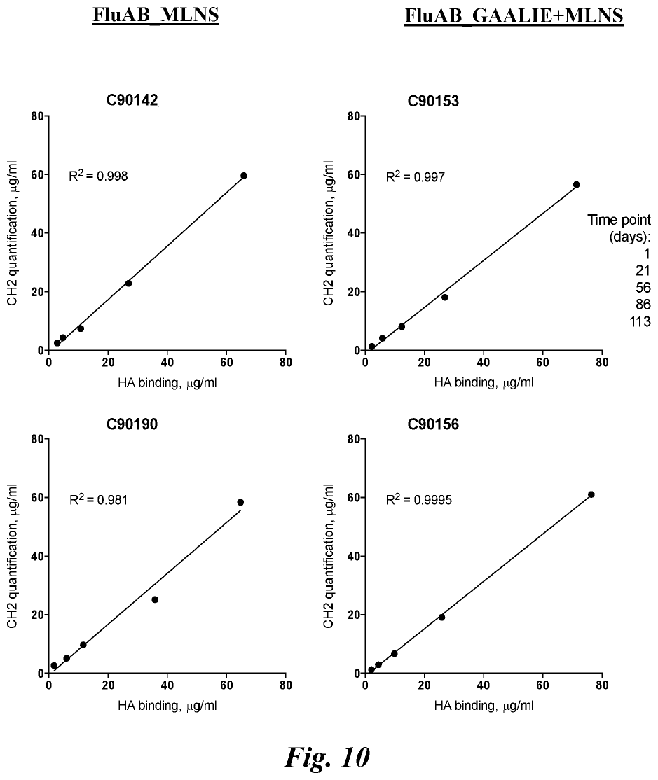

Results are shown in FIG. 9. FIG. 9 shows the plasma concentration of antibodies FluAB_GAALIE+MLNS, FluAB_MLNS, and FluAB_wt following intravenous infusion into cynomolgus monkeys. Analysis of cynomolgus plasma samples collected up to 56 days post-dose demonstrated that both, FluAB_MLNS and FluAB_GAALIE+MLNS, had extended in vivo half-lives (t_{1/2}) compared to FluAB_wt. Overall, clearance was similar for FluAB_MLNS and FluAB_GAALIE+MLNS.

In vivo, post-translation modification of antibodies can occur, which may alter the biological activity of antibody drugs. To test in vivo integrity of antibodies FluAB_MLNS and FluAB_GAALIE+MLNS, pharmacokinetic (PK) measurements were extended to days 86 and 113 post-dose. On days 1, 21, 56, 86, 113 post-dose, human mAb was quantified (i) using the ELISA method as described above as well as another ELISA method measuring the total amount of drug in plasma. To measure total drug (antibody) in cynomolgus plasma, ELISA plates were coated with mouse anti-human IgG (human CH2 domain-specific) and incubated over night at 4° C. After washing and blocking the plates, standards and samples were added and incubated for 1 h at RT. Detection was achieved by addition of goat anti-human IgG HRP, followed by SureBlue TMB Substrate to develop the reaction, which was stopped by adding HCl; absorbance was measured at 450 nm.

Results are shown in FIG. 10. Both quantification methods resulted in similar concentrations of antibodies FluAB_MLNS and FluAB_GAALIE+MLNS, respectively, in cynomolgus plasma. Linear regression analysis further confirmed the integrity of antibodies FluAB_MLNS and FluAB_GAALIE+MLNS up to 113 days in vivo.

In summary, these data show that antibodies FluAB_MLNS and FluAB_GAALIE+MLNS exhibit a similarly enhanced half-life in vivo. Accordingly, the “GAALIE”-mutation of antibodies of the disclosure does not compromise the increased half-life mediated by a half-life increasing Fc mutation.

### Example 9

### The “GAALIE”-Mutation in Antibodies of the Disclosure Mediates Reduced Complement-Dependent Cytotoxicity

Next, complement-dependent cytotoxicity (CDC) was investigated in a CDC assay.

### Preparation of Influenza Virus Infected Target Cells:

To prepare the influenza virus infected target cells, MDCK cells were seeded in a T25 flask at 3.2×106 cells/flask in 5 ml of MEM supplemented with 10% FBS, glutamine, and antibiotics and incubated overnight at 37° C. with 5% C02. Cells were infected with A/California/07/2009 (H1N1) at an MOI of 5 in a total volume of 0.8 ml per flask for one hour at 37° C. After viral adsorption, 4.2 ml of MEM supplemented with 10% FBS, glutamine and antibiotics was added and cells were incubated for 20 hours at 37° C. with 5% CO2. Infected cells were washed with PBS, detached by trypsin digestion using 0.25% Trypson-EDTA, and collected. Infected cells were washed two times with 4 ml of AIM-V medium and centrifuged at 350×g for 4 minutes. Infected viable cells were counted using a Neubauer chamber and adjusted to 1.0×10\hat{ }6 cells/ml for complement pre-adsorption, or adjusted to 1.0×10\hat{ }6 cells/well and stored at 37° C. for use as target cells.

### Preparation of Complement:

Guinea pig low tox complement was reconstituted with 1 ml of cold AIM-V medium. Complement was pre-adsorbed with infected MDCK cells to remove guinea pig antibodies directed against MDCK cells or influenza proteins. In brief, 1.0×10∂pelleted infected MDCK cells were mixed with 1 ml of reconstituted complement and incubated on ice for 45 minutes. After incubation, the mixture was centrifuged for 5 minutes at 400×g at 4° C., the supernatant containing the pre-adsorbed complement was collected, and 4 ml of cold AIM-V was added, creating a 1:6 complement to medium solution.

### Determination of Complement-Mediated Killing:

MAbs were serially diluted in AIM-V medium 4-fold from 100 μg/ml to 0.006 μg/ml. Infected target cell were added to a flat bottom 96-well plate at 5.0×104 cells/well in 50 μl, along with 50 μl of the diluted mAb, and the mixture was incubated for 10 minutes at room temperature. After incubation, 50 μl of the pre-adsorbed guinea pig complement diluted in AIM-V medium was added to each well. Control wells were also included that were used to measure maximal lysis (containing target cells and complement with 50 μl of 2% Triton X-100) and spontaneous lysis (containing target cells and complement only). Plates were incubated for 3 hours at 37° C. with 5% C02. Cell death was quantified by measuring LDH release using LDH detection kit according to the manufacturer's instructions. In brief, plates were centrifuged for 5 minutes at 400×g, and 50 μl of supernatant was transferred to a flat bottom 96-well plate. LDH reagent was prepared and 50 μl of the resulting solution was added to each well. Using a kinetic protocol the absorbance at 490 nm and 650 nm was measured once a minute for 8 minutes. The percent specific lysis was determined by applying the following formula: (specific release−spontaneous release)/(maximum release−spontaneous release)×100.

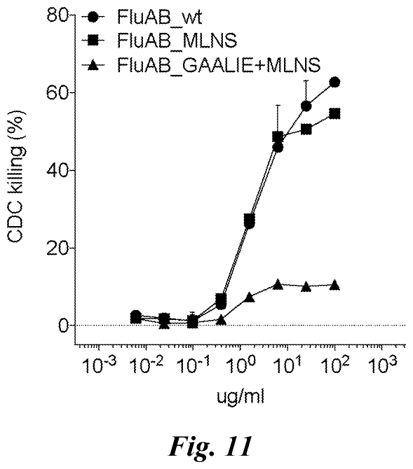

Results are shown in FIG. 11. In contrast to antibodies without the “GAALIE”-mutation, FluAB_GAALIE+MLNS shows a remarkable decrease in complement-dependent cytotoxicity. Accordingly, not only binding to complement is abolished (as shown in Example 4), but also CDC killing. Without being bound to any theory, it is thought that the strongly reduced CDC binding and killing in antibodies with the “GAALIE”-mutation, as shown herein, advantageously abrogates the risk of enhanced disease, because complement is known deposit on immune-complexes resulting in an enhancement of the disease.

TABLE OF
SEQUENCES AND SEQ ID NUMBERS (SEQUENCE LISTING):

| | | |
|---|---|---|
| SEQ ID NO | Sequence | Remarks |

FluAB_GAALIE

| | | |
|---|---|---|
| SEQ ID NO: 1 | SYNAVWN | CDRH1 |
| SEQ ID NO: 2 | RTYYRSGWYNDYAESVKS | CDRH2 |
| SEQ ID NO: 3 | SGHITVFGVNVDAFDM | CDRH3 |
| SEQ ID NO: 4 | RTSQSLSSYTH | CDRL1 |
| SEQ ID NO: 5 | AASSRGS | CDRL2 |
| SEQ ID NO: 6 | QQSRT | CDRL3 |
| SEQ ID NO: 7 | QVQLQQSGPGLVKPSQTLSLTCAISGDSVSSYNA | VH |
| | VWNWIRQSPSRGLEWLGRTYYRSGWYNDYAES | |
| | VKSRITINPDTSKNQFSLQLNSVTPEDTAVYYCAR | |
| | SGHITVFGVNVDAFDMWGQGTMVTVSS | |
| SEQ ID NO: 8 | DIQMTQSPSSLSASVGDRVTITCRTSQSLSSYTH | VL |
| | WYQQKPGKAPKLLIYAASSRGSGVPSRFSGSGS | |
| | GTDFTLTISSLQPEDFATYYCQQSRTFGQGTKVEI | |
| | K | |
| SEQ ID NO: 9 | QVQLQQSGPGLVKPSQTLSLTCAISGDSVSSYNA | Heavy chain |
| | VWNWIRQSPSRGLEWLGRTYYRSGWYNDYAES | |
| | VKSRITINPDTSKNQFSLQLNSVTPEDTAVYYCAR | |
| | SGHITVFGVNVDAFDMWGQGTMVTVSSASTKGP | |
| | SVFPLAPSSKSTSGGTAALGCLVKDYFPEPVTVS | |
| | WNSGALTSGVHTFPAVLQSSGLYSLSSVVTVPSS | |
| | SLGTQTYICNVNHKPSNTKVDKRVEPKSCDKTHT | |
| | CPPCPAPELLAGPSVFLFPPKPKDTLMISRTPEVT | |
| | CVVVDVSHEDPEVKFNWYVDGVEVHNAKTKPRE | |
| | EQYNSTYRVVSVLTVLHQDWLNGKEYKCKVSNKA | |
| | LPLPEEKTISKAKGQPREPQVYTLPPSREEMTKN | |
| | QVSLTCLVKGFYPSDIAVEWESNGQPENNYKTTP | |
| | PVLDSDGSFFLYSKLTVDKSRWQQGNVFSCSVM | |
| | HEALHNHYTQKSLSLSPGK | |
| SEQ ID NO: 10 | DIQMTQSPSSLSASVGDRVTITCRTSQSLSSYTH | Light chain |
| | WYQQKPGKAPKLLIYAASSRGSGVPSRFSGSGS | |
| | GTDFTLTISSLQPEDFATYYCQQSRTFGQGTKVEI | |
| | KRTVAAPSVFIFPPSDEQLKSGTASVVCLLNNFYP | |
| | REAKVQWKVDNALQSGNSQESVTEQDSKDSTYS | |
| | LSSTLTLSKADYEKHKVYACEVTHQGLSSPVTKSF | |
| | NRGEC | |

FluAB_wt

| | | |
|---|---|---|
| SEQ ID NO: 11 | QVQLQQSGPGLVKPSQTLSLTCAISGDSVSSYNA | Heavy chain |
| | VWNWIRQSPSRGLEWLGRTYYRSGWYNDYAES | |
| | VKSRITINPDTSKNQFSLQLNSVTPEDTAVYYCAR | |
| | SGHITVFGVNVDAFDMWGQGTMVTVSSASTKGP | |
| | SVFPLAPSSKSTSGGTAALGCLVKDYFPEPVTVS | |
| | WNSGALTSGVHTFPAVLQSSGLYSLSSVVTVPSS | |
| | SLGTQTYICNVNHKPSNTKVDKRVEPKSCDKTHT | |
| | CPPCPAPELLGGPSVFLFPPKPKDTLMISRTPEVT | |
| | CVVVDVSHEDPEVKFNWYVDGVEVHNAKTKPRE | |
| | EQYNSTYRVVSVLTVLHQDWLNGKEYKCKVSNK | |
| | ALPAPIEKTISKAKGQPREPQVYTLPPSREEMTKN | |
| | QVSLTCLVKGFYPSDIAVEWESNGQPENNYKTTP | |
| | PVLDSDGSFFLYSKLTVDKSRWQQGNVFSCSVM | |
| | HEALHNHYTQKSLSLSPGK | |

FluAB_MLNS

| | | |
|---|---|---|
| SEQ ID NO: 12 | QVQLQQSGPGLVKPSQTLSLTCAISGDSVSSYNA | Heavy chain |
| | VWNWIRQSPSRGLEWLGRTYYRSGWYNDYAES | |
| | VKSRITINPDTSKNQFSLQLNSVTPEDTAVYYCAR | |
| | SGHITVFGVNVDAFDMWGQGTMVTVSSASTKGP | |
| | SVFPLAPSSKSTSGGTAALGCLVKDYFPEPVTVS | |
| | WNSGALTSGVHTFPAVLQSSGLYSLSSVVTVPSS | |
| | SLGTQTYICNVNHKPSNTKVDKRVEPKSCDKTHT | |
| | CPPCPAPELLGGPSVFLFPPKPKDTLMISRTPEVT | |
| | CVVVDVSHEDPEVKFNWYVDGVEVHNAKTKPRE | |
| | EQYNSTYRVVSVLTVLHQDWLNGKEYKCKVSNK | |
| | ALPAPIEKTISKAKGQPREPQVYTLPPSREEMTKN | |
| | QVSLTCLVKGFYPSDIAVEWESNGQPENNYKTTP | |
| | PVLDSDGSFFLYSKLTVDKSRWQQGNVFSCSVL | |
| | HEALHSHYTQKSLSLSPGK | |

FluAB_MLNS+GAALIE

| | | |
|---|---|---|
| SEQ ID NO: 13 | QVQLQQSGPGLVKPSQTLSLTCAISGDSVSSYNA | Heavy chain |
| | IVWNWIRQSPSRGLEWLGRTYYRSGWYNDYAES | |
| | VKSRITINPDTSKNQFSLQLNSVTPEDTAVYYCAR | |
| | SGHITVFGVNVDAFDMWGQGTMVTVSSASTKGP | |
| | SVFPLAPSSKSTSGGTAALGCLVKDYFPEPVTVS | |
| | WNSGALTSGVHTFPAVLQSSGLYSLSSVVTVPSS | |
| | SLGTQTYICNVNHKPSNTKVDKRVEPKSCDKTHT | |
| | CPPCPAPELLAGPSVFLFPPKPKDTLMISRTPEVT | |
| | CVVVDVSHEDPEVKFNWYVDGVEVHNAKTKPRE | |
| | JEQYNSTYRVVSVLTVLHQDWLNGKEYKCKVSNKA | |
| | LPLPEEKTISKAKGQPREPQVYTLPPSREEMTKN | |
| | QVSLTCLVKGFYPSDIAVEWESNGQPENNYKTTP | |
| | PVLDSDGSFFLYSKLTVDKSRWQQGNVFSCSVLH | |
| | EALHSHYTQKSLSLSPGK | |
| SEQ ID NO: 14 | CAAGTTCAGCTGCAGCAGAGCGGCCCCGGTCT | Heavy chain |
| | GGTGAAGCCTAGCCAGACTCTGTCTTTAACTTG | |
| | CGCCATCTCCGGCGACAGCGTGAGCAGCTACA | |
| | ACGCCGTCTGGAACTGGATTCGTCAGAGCCCTA | |
| | GCAGAGGTTTAGAGTGGCTGGGTCGTACTTACT | |
| | ATCGTTCCGGCTGGTACAACGACTACGCCGAGA | |
| | GCGTGAAGTCTCGTATCACTATCAACCCCGATA | |
| | CTAGCAAGAACCAGTTCTCTTTACAGCTGAACA | |
| | GCGTGACTCCCGAAGACACTGCCGTGTACTACT | |
| | GCGCTCGTAGCGGCCACATCACTGTGTTCGGC | |
| | GTGAATGTGGACGCCTTCGACATGTGGGGCCA | |
| | AGGTACTATGGTCACTGTGAGCAGCGCTAGCAC | |
| | CAAGGGCCCATCGGTCTTCCCCCTGGCACCCT | |
| | CCTCCAAGAGCACCTCTGGGGGCACAGCGGCC | |
| | CTGGGCTGCCTGGTCAAGGACTACTTCCCCGAA | |
| | CCGGTGACGGTGTCGTGGAACTCAGGCGCCCT | |
| | GACCAGCGGCGTGCACACCTTCCCGGCCGTCC | |
| | TACAGTCCTCAGGACTCTACTCCCTCAGCAGCG | |
| | TGGTGACCGTGCCCTCCAGCAGCTTGGGCACC | |
| | CAGACCTACATCTGCAACGTGAATCACAAGCCC | |
| | AGCAACACCAAGGTGGACAAGCGGGTTGAGCC | |
| | CAAATCTTGTGACAAAACTCACACATGCCCACC | |
| | GTGCCCAGCACCTGAACTCCTGGCCGGACCGT | |
| | CAGTCTTCCTCTTCCCCCCAAAACCCAAGGACA | |
| | CCCTCATGATCTCCCGGACCCCTGAGGTCACAT | |
| | GCGTGGTGGTGGACGTGAGCCACGAAGACCCT | |
| | GAGGTCAAGTTCAACTGGTACGTGGACGGCGT | |
| | GGAGGTGCATAATGCCAAGACAAAGCCGCGGG | |
| | AGGAGCAGTACAACAGCACGTACCGTGTGGTCA | |
| | GCGTCCTCACCGTCCTGCACCAGGACTGGCTG | |
| | AATGGCAAGGAGTACAAGTGCAAGGTCTCCAAC | |
| | AAAGCCCTCCCACTGCCCGAGGAGAAAACCATC | |
| | TCCAAAGCCAAAGGGCAGCCCCGAGAACCACA | |
| | GGTGTACACCCTGCCCCCATCCCGGGAGGAGA | |
| | TGACCAAGAACCAGGTCAGCCTGACCTGCCTG | |
| | GTCAAAGGCTTCTATCCCAGCGACATCGCCGTG | |
| | GAGTGGGAGAGCAATGGGCAGCCGGAGAACAA | |
| | CTACAAGACCACGCCTCCCGTGCTGGACTCCGA | |
| | CGGCTCCTTCTTCCTCTACAGCAAGCTCACCGT | |
| | GGACAAGAGCAGGTGGCAGCAGGGGAACGTCT | |
| | TCTCATGCTCCGTGCTGCATGAGGCTCTGCACA | |
| | GCCACTACACGCAGAAGAGCCTCTCCCTGTCTC | |
| | CGGGTAAA | |
| SEQ ID NO: 15 | GACATCCAGATGACTCAGAGCCCTTCCTCTTTA | Light chain |
| | AGCGCTAGCGTGGGCGATAGGGTCACTATCACT | |
| | TGTCGTACTAGCCAGTCTTTAAGCTCCTACACTC | |
| | ACTGGTACCAGCAGAAGCCCGGTAAGGCCCCT | |
| | AAGCTGCTGATCTACGCTGCCAGCAGCAGAGG | |
| | CAGCGGAGTGCCTAGCAGATTTAGCGGCAGCG | |
| | GTAGCGGCACTGACTTCACTCTGACAATCAGCT | |
| | CTTTACAGCCCGAAGACTTCGCCACTTACTACT | |
| | GCCAGCAGTCTCGTACTTTCGGCCAAGGTACTA | |
| | AGGTGGAGATCAAGCGTACGGTGGCTGCACCA | |
| | TCTGTCTTCATCTTCCCGCCATCTGATGAGCAGT | |
| | TGAAATCTGGAACTGCCTCTGTTGTGTGCCTGC | |
| | TGAATAACTTCTATCCCAGAGAGGCCAAAGTAC | |
| | AGTGGAAGGTGGATAACGCCCTCCAATCGGGTA | |
| | ACTCCCAGGAGAGTGTCACAGAGCAGGACAGC | |
| | AAGGACAGCACCTACAGCCTCAGCAGCACCCT | |
| | GACGCTGAGCAAAGCAGACTACGAGAAACACAA | |
| | AGTCTACGCCTGCGAAGTCACCCATCAGGGCCT | |
| | GAGCTCGCCCGTCACAAAGAGCTTCAACAGGG | |
| | GAGAGTGT | |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An antibody comprising a heavy chain comprising CDR1, CDR2, and CDR3 sequences as set forth in SEQ ID NO: 1, SEQ ID NO: 2, and SEQ ID NO: 3, respectively; a light chain comprising CDR1, CDR2, and CDR3 sequences as set forth in SEQ ID NO: 4, SEQ ID NO: 5, and SEQ ID NO: 6, respectively; and a G236A mutation, A330L mutation, and I332E mutation in the constant region of the heavy chain.

2. The antibody of claim 1, wherein the antibody does not comprise S239D mutation in the constant region of the heavy chain.

3. The antibody of claim 1 or 2, wherein the antibody binds to hemagglutinin of an influenza A virus.

4. The antibody of any one of claims 1-3, wherein the antibody neutralizes infection with an influenza A virus.

5. The antibody of any one of claims 1-4, wherein the antibody comprises a half-life increasing mutation in the constant region of the heavy chain.

6. The antibody of claim 5, wherein the antibody comprises a M428L mutation and N434S mutation in the constant region of the heavy chain.

7. The antibody of any one of the previous claims, wherein the antibody is a human antibody.

8. The antibody of any one of the previous claims, wherein the antibody is a monoclonal antibody.

9. The antibody of any one of the previous claims, wherein the antibody is an IgG type.

10. The antibody of claim 9, wherein the antibody is an IgG1 type.

11. The antibody of any one of the previous claims, wherein the light chain of the antibody is a kappa light chain.

12. The antibody of any one of the previous claims, wherein the antibody comprises a heavy chain variable region comprising an amino acid sequence having at least 70% identity to SEQ ID NO: 7 and a light chain variable region comprising an amino acid sequence having at least 70% identity to SEQ ID NO: 8, wherein the CDR sequences as defined in claim 1 are maintained.

13. The antibody of any one of the previous claims, wherein the antibody comprises a heavy chain variable region comprising an amino acid sequence having at least 75% identity to SEQ ID NO: 7 and a light chain variable region comprising an amino acid sequence having at least 75% identity to SEQ ID NO: 8, wherein the CDR sequences as defined in claim 1 are maintained.

14. The antibody of any one of the previous claims, wherein the antibody comprises a heavy chain variable region comprising an amino acid sequence having at least 80% identity to SEQ ID NO: 7 and a light chain variable region comprising an amino acid sequence having at least 80% identity to SEQ ID NO: 8, wherein the CDR sequences as defined in claim 1 are maintained.

15. The antibody of any one of the previous claims, wherein the antibody comprises a heavy chain variable region comprising an amino acid sequence having at least 85% identity to SEQ ID NO: 7 and a light chain variable region comprising an amino acid sequence having at least 85% identity to SEQ ID NO: 8, wherein the CDR sequences as defined in claim 1 are maintained.

16. The antibody of any one of the previous claims, wherein the antibody comprises a heavy chain variable region comprising an amino acid sequence having at least 90% identity to SEQ ID NO: 7 and a light chain variable region comprising an amino acid sequence having at least 90% identity to SEQ ID NO: 8, wherein the CDR sequences as defined in claim 1 are maintained.

17. The antibody of any one of the previous claims, wherein the antibody comprises a heavy chain variable region comprising an amino acid sequence having at least 95% identity to SEQ ID NO: 7 and a light chain variable region comprising an amino acid sequence having at least 95% identity to SEQ ID NO: 8, wherein the CDR sequences as defined in claim 1 are maintained.

18. The antibody of any one of the previous claims, wherein the antibody comprises a heavy chain variable region comprising an amino acid sequence as set forth in SEQ ID NO: 7 and a light chain variable region comprising the amino acid sequence as set forth in SEQ ID NO: 8, wherein the CDR sequences as defined in claim 1 are maintained.

19. The antibody of any one of the previous claims, wherein the antibody comprises a CH2 region that does not comprise further mutation in addition to G236A, A330L and I332E.

20. The antibody of any one of the previous claims, wherein the antibody comprises an Fc region that does not comprise further mutation in addition to G236A, A330L and I332E and, optionally, M428L and N434S.

21. The antibody of any one of the previous claims, wherein the antibody comprises a heavy chain comprising an amino acid sequence as set forth in SEQ ID NO: 9 and a light chain comprising an amino acid sequence as set forth in SEQ ID NO: 10.

22. The antibody of any one of the previous claims, wherein the antibody comprises a heavy chain comprising an amino acid sequence as set forth in SEQ ID NO: 13 and a light chain comprising an amino acid sequence as set forth in SEQ ID NO: 10.

23. The antibody of any one of the previous claims, wherein the antibody has a heavy chain consisting of an amino acid sequence as set forth in SEQ ID NO: 9 and a light chain consisting of an amino acid sequence as set forth in SEQ ID NO: 10.

24. The antibody of any one of the previous claims, wherein the antibody has a heavy chain consisting of an amino acid sequence as set forth in SEQ ID NO: 13 and a light chain consisting of an amino acid sequence as set forth in SEQ ID NO: 10.

25. The antibody of any one of the previous claims for use in prophylaxis or treatment of infection with influenza A virus.

26. A nucleic acid molecule comprising a polynucleotide encoding the antibody of any one of claims 1-24.

27. The nucleic acid molecule of claim 26, wherein the polynucleotide comprises deoxyribonucleic acid (DNA) or ribonucleic acid (RNA), wherein the RNA optionally comprises messenger RNA (mRNA).

28. The nucleic acid molecule of claim 26 or 27, comprising a modified nucleoside, a cap-1 structure, a cap-2 structure, or any combination thereof.

29. The nucleic acid molecule of claim 28, wherein the polynucleotide comprises a pseudouridine, a N6-methyladenonsine, a 5-methylcytidine, a 2-thiouridine, or any combination thereof.

30. The nucleic acid molecule of claim 29, wherein the pseudouridine comprises N1-methylpseudouridine.

31. A vector comprising the nucleic acid molecule of any one of claims 26-30.

32. A cell expressing the antibody of any one of claims 1-24, or comprising the vector of claim 31.

33. A pharmaceutical composition comprising the antibody of any one of claims 1-24, the nucleic acid molecule of any one of claims 26-30, the vector of claim 31, or the cell of claim 32, and, optionally, a pharmaceutically acceptable diluent or carrier.

34. A composition comprising the nucleic acid molecule of any one of claims 26-30 or the vector of claim 31 encapsulated in a carrier molecule, wherein the carrier molecule optionally comprises a lipid, a lipid-derived delivery vehicle, such as a liposome, a solid lipid nanoparticle, an oily suspension, a submicron lipid emulsion, a lipid microbubble, an inverse lipid micelle, a cochlear liposome, a lipid microtubule, a lipid microcylinder, lipid nanoparticle (LNP), or a nanoscale platform.

35. Use of the antibody of any one of claims 1-24, the nucleic acid molecule of any one of claims 26-30, the vector of claim 31, the cell of claim 32, the pharmaceutical composition of claim 33, or the composition of claim 34 in the manufacture of a medicament for prophylaxis, treatment or attenuation of influenza A virus infection.

36. The antibody of any one of claims 1-24, the nucleic acid molecule of any one of claims 26-30, the vector of claim 31, the cell of claim 32, the pharmaceutical composition of claim 33, or the composition of claim 34 for use in prophylaxis or treatment of infection with influenza A virus.

37. A method of reducing influenza A virus infection, or lowering the risk of influenza A virus infection, comprising: administering to a subject in need thereof, a therapeutically effective amount of the antibody of any one of claims 1-24, the nucleic acid molecule of any one of claims 26-30, the vector of claim 31, the cell of claim 32, the pharmaceutical composition of claim 33, or the composition of claim 34.


SEQUENCE LISTING (15 sequences)

SEQ ID NO: 1 - protein, 7 aa, Artificial Sequence
  Synthetic Sequence CDRH1
SYNAVWN

SEQ ID NO: 2 - protein, 18 aa, Artificial Sequence
  Synthetic Sequence CDRH2
RTYYRSGWYNDYAESVKS

SEQ ID NO: 3 - protein, 16 aa, Artificial Sequence
  Synthetic Sequence CDRH3
SGHITVFGVNVDAFDM

SEQ ID NO: 4 - protein, 11 aa, Artificial Sequence
  Synthetic Sequence CDRL1
RTSQSLSSYTH

SEQ ID NO: 5 - protein, 7 aa, Artificial Sequence
  Synthetic Sequence CDRL2
AASSRGS

SEQ ID NO: 6 - protein, 5 aa, Artificial Sequence
  Synthetic Sequence CDRL3
QQSRT

SEQ ID NO: 7 - protein, 128 aa, Artificial Sequence
  Synthetic Sequence VH
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSS

SEQ ID NO: 8 - protein, 103 aa, Artificial Sequence
  Synthetic Sequence VL
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIK

SEQ ID NO: 9 - protein, 458 aa, Artificial Sequence
  Synthetic Sequence Heavy chain
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 210 aa, Artificial Sequence
  Synthetic Sequence Light chain
DIQMTQSPSSLSASVGDRVTITCRTSQSLSSYTHWYQQKPGKAPKLLIYAASSRGSGVPSRFSGSGSGTDFTLTISSLQPEDFATYYCQQSRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 458 aa, Artificial Sequence
  Synthetic Sequence Heavy chain
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 458 aa, Artificial Sequence
  Synthetic Sequence Heavy chain
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 13 - protein, 458 aa, Artificial Sequence
  Synthetic Sequence Heavy chain
QVQLQQSGPGLVKPSQTLSLTCAISGDSVSSYNAVWNWIRQSPSRGLEWLGRTYYRSGWYNDYAESVKSRITINPDTSKNQFSLQLNSVTPEDTAVYYCARSGHITVFGVNVDAFDMWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 14 - DNA, 1374 bp, Artificial Sequence
  Synthetic heavy chain sequence
caagttcagctgcagcagagcggccccggtctggtgaagcctagccagactctgtctttaacttgcgccatctccggcgacagcgtgagcagctacaacgccgtctggaactggattcgtcagagccctagcagaggtttagagtggctgggtcgtacttactatcgttccggctggtacaacgactacgccgagagcgtgaagtctcgtatcactatcaaccccgatactagcaagaaccagttctctttacagctgaacagcgtgactcccgaagacactgccgtgtactactgcgctcgtagcggccacatcactgtgttcggcgtgaatgtggacgccttcgacatgtggggccaaggtactatggtcactgtgagcagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggccgtcctacagtcctcaggactctactccctcagcagcgtggtgaccgtgccctccagcagcttgggcacccagacctacatctgcaacgtgaatcacaagcccagcaacaccaaggtggacaagcgggttgagcccaaatcttgtgacaaaactcacacatgcccaccgtgcccagcacctgaactcctggccggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccactgcccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcacagccactacacgcagaagagcctctccctgtctccgggtaaa

SEQ ID NO: 15 - DNA, 630 bp, Artificial Sequence
  Synthetic light chain sequence
gacatccagatgactcagagcccttcctctttaagcgctagcgtgggcgatagggtcactatcacttgtcgtactagccagtctttaagctcctacactcactggtaccagcagaagcccggtaaggcccctaagctgctgatctacgctgccagcagcagaggcagcggagtgcctagcagatttagcggcagcggtagcggcactgacttcactctgacaatcagctctttacagcccgaagacttcgccacttactactgccagcagtctcgtactttcggccaaggtactaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt